SEQUENCE LISTING (32 sequences)

SEQ ID NO: 1 - DNA, 25 bp, Artificial Sequence
  aptamer
ccaguggguugaaggaaaguaacag

SEQ ID NO: 2 - DNA, 103 bp, Artificial Sequence
  riboswitch
gaaggauccaguggguugaaggaaaguaacagauccuccaggaugcgacccgguuaucgaauuaaggagguaaaaaaugcaagucgaccugcuggauccaaac

SEQ ID NO: 3 - DNA, 103 bp, Artificial Sequence
  riboswitch
gaaggauccaguggguugaaggaaaguaacagauccuuuaggaugcgacccgguuaucgaauuaaggagguaaaaaaugcaagucgaccugcuggauccaaac

SEQ ID NO: 4 - DNA, 103 bp, Artificial Sequence
  riboswitch
gaaggauccaguggguugaaggaaaguaacagauccuccaggaugcgacccgguuaucgaauuaaggagguaaaaaaugcaagucgaccugcucgauccaaac

SEQ ID NO: 5 - DNA, 102 bp, Artificial Sequence
  riboswitch
gaggauccaguggguugaaggaaaguaacagauccuucaggaugcgacccgguuaucgaauuaaggagguaaaaaaugcaagucgaccugcuggauccaaac

SEQ ID NO: 6 - DNA, 100 bp, Artificial Sequence
  riboswitch
gacgauccaguggguugaaggaaaguaacagaucguucaggaugcgacgcgcuuggaauuaaggagguaaaaaaugcaagucgaccugcuggauccaaac

SEQ ID NO: 7 - DNA, 37 bp, Artificial Sequence
  aptamer
uacgauccaguggguugaaggaaaguaacagaucgua

SEQ ID NO: 8 - DNA, 37 bp, Artificial Sequence
  aptamer
aucguaccaguggguugaaggaaaguaacaguacgau

SEQ ID NO: 9 - DNA, 86 bp, Artificial Sequence
  aptamer
uagggaagagaaggacauaugauccaguggguugaaggaaaguaacagaucguaugagcgggguugacuaguacaugaccacuuga

SEQ ID NO: 10 - DNA, 37 bp, Artificial Sequence; identical to SEQ ID NO: 7
  aptamer

SEQ ID NO: 11 - DNA, 86 bp, Artificial Sequence
  template
  misc_feature (14)..(63): n is a, c, g, or t
tagggaagagaaggacatatgatnnnnnnnnnnnnnnnnnnnnnnnnnnnnnnnnnnnnnnnnttgactagtacatgaccacttga

SEQ ID NO: 12 - DNA, 23 bp, Artificial Sequence
  primer
tcaagtggtcatgtactagtcaa

SEQ ID NO: 13 - DNA, 44 bp, Artificial Sequence
  primer
ttcaggtaatacgactcactatagggaagagaaggacatatgat

SEQ ID NO: 14 - DNA, 86 bp, Artificial Sequence
  template
tagggaagagaaggacatatgatccagtgggttgaaggaaagtaacagatcgtatgagcggggttgactagtacatgaccacttga

SEQ ID NO: 15 - DNA, 37 bp, Artificial Sequence; identical to SEQ ID NO: 7
  aptamer

SEQ ID NO: 16 - DNA, 10 bp, Artificial Sequence
  quencher DNA
ctggatcgta

SEQ ID NO: 17 - DNA, 11 bp, Artificial Sequence
  quencher DNA
actggatcgta

SEQ ID NO: 18 - DNA, 12 bp, Artificial Sequence
  quencher DNA
cactggatcgta

SEQ ID NO: 19 - DNA, 13 bp, Artificial Sequence
  quencher DNA
ccactggatcgta

SEQ ID NO: 20 - DNA, 14 bp, Artificial Sequence
  quencher DNA
cccactggatcgta

SEQ ID NO: 21 - DNA, 15 bp, Artificial Sequence
  quencher DNA
acccactggatcgta

SEQ ID NO: 22 - DNA, 17 bp, Artificial Sequence
  quencher DNA
caacccactggatcgta

SEQ ID NO: 23 - DNA, 792 bp, Artificial Sequence
  riboswitch construct
taatacgactcactatagactcacatacgagatatatagaaggaggtaataaatggcaatcattaaggagttcatgagattcaaagttcacatggaaggttctgtaaatggacatgaatttgaaatagaaggtgaaggagaaggaaggccttatgaaggaacccaaaccgcgaagctaaaagttactaagggtggcccattaccatttgcatgggatatccttagccctcaattcatgtatgggtcaaaggcttatgtcaagcaccccgccgacattccagactatctaaagttatcttttcccgaagggtttaagtgggagcgtgtgatgaacttcgaagacggtggcgtggtaacagtgactcaggattcgtccctgcaagatggtgaatttatctacaaagtcaaattaagaggaactaactttccatctgacggcccggttatgcaaaaaaagacaatgggctgggaggcctcctcagaacgaatgtaccctgaagatggtgccttgaagggtgagattaaacaaagattgaaattgaaagatggtggacattatgacgctgaggttaaaacgacatacaaagctaagaaacctgtccagctcccaggtgcttacaatgtaaatataaaacttgatattacatcacataatgaagattatacgatagttgaacaatacgaaagggctgaggggagacatagtactggtggcatggatgaactatacaaaggttctggtaccgcataactagcataacccctctctaaacggaggggtttgaattctat

SEQ ID NO: 24 - DNA, 839 bp, Artificial Sequence
  riboswitch construct
taatacgactcactatagaaggatccagtgggttgaaggaaagtaacagatcctccaggatgcgacccggttatcgaattaaggaggtaaaaaatgcaagtcgacctgctggatccaaacatgagattcaaagttcacatggaaggttctgtaaatggacatgaatttgaaatagaaggtgaaggagaaggaaggccttatgaaggaacccaaaccgcgaagctaaaagttactaagggtggcccattaccatttgcatgggatatccttagccctcaattcatgtatgggtcaaaggcttatgtcaagcaccccgccgacattccagactatctaaagttatcttttcccgaagggtttaagtgggagcgtgtgatgaacttcgaagacggtggcgtggtaacagtgactcaggattcgtccctgcaagatggtgaatttatctacaaagtcaaattaagaggaactaactttccatctgacggcccggttatgcaaaaaaagacaatgggctgggaggcctcctcagaacgaatgtaccctgaagatggtgccttgaagggtgagattaaacaaagattgaaattgaaagatggtggacattatgacgctgaggttaaaacgacatacaaagctaagaaacctgtccagctcccaggtgcttacaatgtaaatataaaacttgatattacatcacataatgaagattatacgatagttgaacaatacgaaagggctgaggggagacatagtactggtggcatggatgaactatacaaaggttctggtaccgcataactagcataacccctctctaaacggaggggtttgaattctat

SEQ ID NO: 25 - DNA, 103 bp, Artificial Sequence
  riboswitch construct
gaaggatccagtgggttgaaggaaagtaacagatcctttaggatgcgacccggttatcgaattaaggaggtaaaaaatgcaagtcgacctgctggatccaaac

SEQ ID NO: 26 - DNA, 103 bp, Artificial Sequence
  riboswitch construct
gaaggatccagtgggttgaaggaaagtaacagatcctccaggatgcgacccggttatcgaattaaggaggtaaaaaatgcaagtcgacctgctcgatccaaac

SEQ ID NO: 27 - DNA, 102 bp, Artificial Sequence
  riboswitch construct
gaggatccagtgggttgaaggaaagtaacagatccttcaggatgcgacccggttatcgaattaaggaggtaaaaaatgcaagtcgacctgctggatccaaac

SEQ ID NO: 28 - DNA, 100 bp, Artificial Sequence
  riboswitch construct
gacgatccagtgggttgaaggaaagtaacagatcgttcaggatgcgacgcgcttggaattaaggaggtaaaaaatgcaagtcgacctgctggatccaaac

SEQ ID NO: 29 - DNA, 878 bp, Artificial Sequence
  riboswitch construct
taatacgactcactatagaaggatccagtgggttgaaggaaagtaacagatcctccaggatgcgacccggttatcgaattaaggaggtaaaaaatgcaagtcgacctgctggatccaaacatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataactagcataacccctctctaaacggaggggtttgaattctat

SEQ ID NO: 30 - DNA, 143 bp, Artificial Sequence
  riboswitch construct
gagctgttgacaattaatcatcggctcgtataatgtgtgggaaggatccagtgggttgaaggaaagtaacagatcctccaggatgcgacccggttatcgaattaaggaggtaaaaaatgcaagtcgacctgctggatccaaac

SEQ ID NO: 31 - DNA, 1043 bp, Artificial Sequence
  riboswitch construct
taatacgactcactatagaaggatccagtgggttgaaggaaagtaacagatcctccaggatgcgacccggttatcgaattaaggaggtaaaaaatgcaagtcgacctgctggatccaaacatggattctgatatcaatatcaaaaccggcaccaccgatatcggctccaataccaccgttaaaaccggtgatctggtgacctatgataaagaaaacggtatgcataaaaaagtgttttactcgtttattgacgataaaaaccataacaaaaaactgctggtcatccgcaccaaaggcaccattgcgggtcaataccgtgtgtactccgaagaaggtgcgaacaaaagcggtctggcttggccgtctgcctttaaagtgcagctgcaactgccggataatgaagtggcgcagatttcagattattatccgcgtaatagcatcgataccaaagaatatatgagtaccctgacctatggttttaatggcaatgttaccggtgatgatacgggtaaaattggcggtctgattggcgccaatgtgtccattggtcatacgctgaaatacgtgcaaccggatttcaaaaccattctggaaagtccgaccgataaaaaagtgggttggaaagttatcttcaacaacatggtgaatcagaactggggtccgtacgatcgcgattcctggaatccggtttatggcaatcagctgtttatgaaaacccgcaacggtagtatgaaagcggcggataattttctggacccgaacaaagcctcaagcctgctgtccagcggttttagcccggattttgccacggttattaccatggatcgcaaagccagcaaacagcagaccaacattgatgtgatctacgaacgtgtgcgtgatgattatcaactgcattggacctcaaccaattggaaaggcaccaataccaaagataaatggacggatcgcagttcagaacgctacaaaattgattgggaaaaagaagaaatgaccaactaactagcataacccctctctaaacggaggggtttgaattctat

SEQ ID NO: 32 - DNA, 87 bp, Artificial Sequence
  aptamer
guagggaagagaaggacauaugauccaguggguugaaggaaaguaacagaucguaugagcgggguugacuaguacaugaccacuuga

(19) United States
(12) Patent Application Publication
(10) Pub. No.: US 2022/0372584 A1
(43) Pub. Date: Nov. 24, 2022

(54) RNA APTAMER THAT SPECIFICALLY BINDS HISTAMINE

(71) Applicant: OKINAWA INSTITUTE OF SCIENCE AND TECHNOLOGY SCHOOL CORPORATION, Okinawa (JP)

(72) Inventors: Yohei YOKOBAYASHI, Okinawa (JP); Mohammed Essameldin Ibrahim Mohammed DWIDAR, Okinawa (JP)

(73) Assignee: OKINAWA INSTITUTE OF SCIENCE AND TECHNOLOGY SCHOOL CORPORATION, Okinawa (JP)

(21) Appl. No.: 17/431,547

(22) PCT Filed: Feb. 18, 2020
(86) PCT No.: PCT/JP2020/006298

§ 371 Date: Aug. 17, 2021

(30) Foreign Application Priority Data
Feb. 18, 2019 (JP) 2019-026510

(51) Int. Cl. C12Q 1/6897; G01N 33/94; G01N 33/58; C12N 15/115; G01N 33/12

(52) U.S. Cl. CPC C12Q 1/6897; G01N 33/9406; G01N 33/582; C12N 15/115; G01N 33/12; C12N 2310/16

(57) ABSTRACT

It is an object of the present invention to provide an RNA aptamer that specifically binds histamine. The present invention is related to an nucleic acid aptamer that binds to histamine, comprising the base sequence (i) or (ii) below: (i) the base sequence of SEQ ID NO: 1; (ii) the base sequence comprising substitution(s), deletion(s), and/or addition(s) of 1 to 3 base(s) in the base sequence of SEQ ID NO: 1.

### TECHNICAL FIELD

The present invention is related to a nucleic acid aptamer that binds to histamine.

### BACKGROUND ART

Histamine is an important metabolite involved in a number of biological processes. For example, if improperly handled, histamine can accumulate in fish and dairy products which contain bacteria that can decarboxylate histidine to produce histamine. Because histamine is not significantly degraded during cooking and high histamine level in food products can cause food poisoning, histamine is routinely monitored in the food industry (Feng et al. 2016). Histamine released from basophils isolated from allergy patients is also used for diagnostic purposes (histamine release test) (Platzer et al. 2005). High level of histamine in plasma and urine is an important indicator of anaphylactic shock (Laroche et al. 1991). Furthermore, measurement of histamine released from or inside cultured cells can benefit immunology and neuroscience (Hu et al. 2007; Kielland et al. 2012).

Currently, the most popular commercial available kits for quantifying histamine in biological samples are based on enzyme-linked immunosorbent assay (ELISA) (Muscarella et al. 2013; Pessatti et al. 2004) or based on colorimetric assay using histamine dehydrogenase (Sato et al. 2005). Although these methods based on an-tibodies and enzymes are well established, dependence on recombinant proteins makes these conventional assays costly and requires careful storage and handling. Instrumental analysis such as those that employ high-performance liquid chromatography (HPLC) (Onal 2007), mass spectrometry (Nei et al. 2017), and surface enhanced Raman spectroscopy (SERS) (Janci et al. 2017) can offer advantages such as high sensitivity, but they are time consuming and require skilled operators. Several small molecule probes for histamine have been reported but their applications have been limited (Kielland et al. 2012; Seto et al. 2010).

Aptamers are single stranded oligonucleotides (DNA or RNA) capable of molecular recognition. Aptamers are usually selected in vitro from a pool of 10¹⁴ to 10′⁵ random sequences through a process called systematic evolution of ligands by exponential enrichment (SELEX) (Codrea et al. 2010; Ruscito and DeRosa 2016). The target can be a small molecule, a protein, or even a whole cell. As molecular recognition elements, aptamers offer several advantages over antibodies (McKeague and Derosa 2012). For example, aptamers cost less to produce and are more stable compared to antibodies. Aptamers of reasonable size (˜40 nt) can be chemically synthesized by well-established and scalable automated solid phase synthesis. While antibodies are highly sensitive to temperature, pH, and salt concentrations, and cannot regain function once denatured, nucleic acid aptamers are chemically stable under most biologically relevant conditions and they can easily be refolded after denaturation. Furthermore, the predictable nature of nucleic acid hybridization has facilitated the development of numerous strategies to engineer aptamers into sensors (Pfeiffer and Mayer 2016).

### SUMMARY OF INVENTION

### Technical Problem

On the other hand, development of aptamers that recognize molecules of practical importance still limit broader applications of aptamers. Unlike the well-established protocols for antibody development, the aptamer selection process remains variable and success or failure of selection and performance of aptamers are highly unpredictable. Selection of good aptamers for small molecules is considered to be more challenging compared to those for macromolecules (McKeague and Derosa 2012; Pfeiffer and Mayer 2016).

Therefore, an object thereof is to provide a nucleic acid aptamer capable of binding to histamine.

### Solution to Problem

The present invention relates to the following;

(1) A nucleic acid aptamer that binds to histamine, comprising the base sequence (i) or (ii) below:

(i) the base sequence of SEQ ID NO: 1;

(ii) the base sequence comprising substitution(s), deletion(s), and/or addition(s) of 1 to 3 base(s) in the base sequence of SEQ ID NO: 1.

(SEQ ID NO: 1)
5′-CCAGUGGGUUGAAGGAAAGUAACAG-3′

(2) The nucleic acid aptamer according to (1), wherein base sequences that can form a double-stranded stem structure through complementary base pairing are added to both ends of the base sequence (i) or (ii).

(3) The nucleic acid aptamer according to (2), wherein stem base sequence 1 is added to the 5′ end or the 3′ end of the base sequence (i) or (ii) and stem base sequence 2 is added to the other end.

5′-UACGAU-3′ (stem base sequence 1)

5′-AUCGUA-3′ (stem base sequence 2)

(4) The nucleic acid aptamer according to any one of (1) to (3), wherein substitution(s) is/are made at least one positon of position 5 (U), position 16 (A), position 17 (A) and position 18 (A) of SEQ ID NO: 1.

(5) The nucleic acid aptamer according to any one of (1) to (4), wherein the binding force to histamine is higher than the binding force to histidine.

(6) The nucleic acid aptamer according to any one of (1) to (5), wherein the dissociation constant for histamine is no more than 5 μM.

(7) The nucleic acid aptamer according to any one of (1) to (6), wherein the dissociation constant for histidine is no less than 40 μM.

(8) The nucleic acid aptamer according to any one of (1) to (7), wherein the nucleic acid is at least one selected from the group consisting of DNA, RNA, and artificial nucleic acid.

(9) The nucleic acid aptamer according to (8), wherein the nucleic acid is at least one selected from the group consisting of L-DNA and L-RNA.

(10) A composition for histamine detection comprising the nucleic acid aptamer according to any one of (1) to (9).

(11) A histamine detection kit comprising the nucleic acid aptamer according to any one of (1) to (9).

(12) A biosensor for detecting histamine, comprising the nucleic acid aptamer according to any one of (1) to (9).

(13) A method for detecting histamine in a subject sample, which method is characterized by using the nucleic acid aptamer according to any one of (1) to (9).

(14) The method for detecting histamine according to (13), which comprises
preparing a complex having a double strand formed of the nucleic acid aptamer according to any one of (1) to (9) and a nucleic acid fragment having a base sequence complementary to a part of the nucleic acid aptamer;
separating the nucleic acid fragment from the nucleic acid aptamer by binding histamine in subject sample with the nucleic acid aptamer; and
detecting cleavage of the double strand by the above separation.

(15) The method for detecting histamine according to (14), wherein a fluorescent substance is bound to the nucleic acid aptamer and a quencher substance is bound to the nucleic acid fragment.

(16) A riboswitch comprising the nucleic acid aptamer according to any one of (1) to (9).

(17) The riboswitch according to (16), which comprises any of the base sequences of SEQ ID NOs: 2 to 6.

(SEQ ID NO: 2)
5′-GAAGGAUCCAGUGGGUUGAAGGAAAGUAACAGAUCCUCCAGGAUGC
GACCCGGUUAUCGAAUUAAGGAGGUAAAAAAUGCAAGUCGACCUGCUGG
AUCCAAAC-3′
(SEQ ID NO: 3)
5′-GAAGGAUCCAGUGGGUUGAAGGAAAGUAACAGAUCCUUUAGGAUGC
GACCCGGUUAUCGAAUUAAGGAGGUAAAAAAUGCAAGUCGACCUGCUGG
AUCCAAAC-3′
(SEQ ID NO: 4)
5′-GAAGGAUCCAGUGGGUUGAAGGAAAGUAACAGAUCCUCCAGGAUGC
GACCCGGUUAUCGAAUUAAGGAGGUAAAAAAUGCAAGUCGACCUGCUCG
AUCCAAAC-3′
(SEQ ID NO: 5)
5′-GAGGAUCCAGUGGGUUGAAGGAAAGUAACAGAUCCUUCAGGAUGCG
ACCCGGUUAUCGAAUUAAGGAGGUAAAAAAUGCAAGUCGACCUGCUGGA
UCCAAAC-3′
(SEQ ID NO: 6)
5′-GACGAUCCAGUGGGUUGAAGGAAAGUAACAGAUCGUUCAGGAUGCG
ACGCGCUUGGAAUUAAGGAGGUAAAAAAUGCAAGUCGACCUGCUGGAUC
CAAAC-3′

(18) A RNA polynucleotide comprising a regulation region corresponding to the riboswitch according to (16) or (17) and a cording region for a protein, wherein the expression of the protein is regulated by the regulation region.

(19) A nucleic acid construct comprising a promoter sequence and a base sequence complementary to the RNA polynucleotide according to (18) operably linked thereto.

In the above base sequences, “U” means “U/T (U or T)”, that is, when the base sequence is DNA base sequence, U (uracil) is replaced by T (thymine).

### Advantageous Effects of the Invention

The nucleic acid aptamer of the present invention has a superior binding force to histamine. Therefore, for example, the nucleic acid aptamer of the present invention is very useful for detection of histamine.

### BRIEF DESCRIPTION OF DRAWINGS

FIG. 1A) Sequence of the A1 aptamer obtained from the first SELEX experiment. (SEQ ID NO: 32) B) Predicted secondary structure of A1 determined by mfold (Zuker 2003). C) Consensus sequence of the reselected sequences from the doped library SELEX based on A1. The originally randomized region (N40) region is shown. Total of 535 sequences that were enriched more than 50-fold after four rounds of selection were compiled using WebLogo (http://weblogo.berkeley.edu/).

FIG. 2A and B) Predicted secondary structures of A1-949 aptamer determined by the online mfold (Zuker 2003). C) ITC results of A1-949 binding with histamine. D) ITC results of A1-949 binding with histidine.

FIG. 3 Schematic illustration of the histamine aptasensor design. Histamine aptamer (orange) is fluorescently labeled at the 5′ end, and it is hybridized with a 3′-quencher modified DNA (purple) with partially complementary sequence to the 5′ end of the aptamer. Binding of histamine to the aptamer displaces the quencher-modified DNA resulting in increased fluorescence.

FIG. 4 Optimization of the aptasensor structure. A) The sequences of the different quencher DNAs (QueN) tested. B) Effect of the length of the quencher strand (QueN) on the aptasensor response.

FIG. 5 Optimization of the aptasensor measurement conditions. The aptamer concentration was 10 nM. A) Optimization of MgCl₂ concentration (250 mM NaCl, 5× Que13). B) Optimization of NaCl concentration (5× Que13, 0 mM MgCl₂). C) Optimization of the quencher strand (Que13) concentration (125 mM NaCl).

FIG. 6 Specificity of the aptasensor. Responses of the atpasensors to histamine, L-histidine, D-histidine, spermidine, and imidazole were measured at 100 μM.

FIG. 7 Dose dependent responses of the aptasensor to histamine and histidine stereoisomers. A) Aptasensor Cy5-A1-949/Que13. B) Aptasensor Cy3-A1-949*/Que13* (L-form).

FIG. 8 Responses of the L-form atpasensor to histamine dissolved in assay buffer and spiked in tuna extract.

FIG. 9 A) Comparing different riboswitch constructs for their ON/OFF ratio and background expression. B) Dynamic range of H2 riboswitch. C) Confirming that the activity of H2 riboswitch is due to aptamer conformation change upon binding to histamine. H2-M8 and H2-M21 constructs have the same sequence as H2 riboswitch with just a single mutation for each in the core aptamer region. Optimum RBS is a genetic construct that has a strong RBS sequence to maximize the expression of mCherry gene. All the genetic constructs have T7 promoter sequence upstream.

FIG. 10 ITC results of the A1 aptamer to histamine (A) and L-histidine (B).

FIG. 11 MST analysis of Cy5-A1-949 aptamer binding to histamine (A) and L-histidine (B).

FIG. 12 PAGE analysis of in vitro transcribed A1-949 aptamer under native and denaturing conditions. A1-949 aptamer (500 μg/ml) was heat denatured at 70° C. in the same buffer used for ITC and then cooled down and kept at room temperature for 20 min. For the native gel, 1 μl of each concentration was mixed with the loading dye, loaded and separated on polyacrylamide gels under native and denaturing conditions, respectively.

FIG. 13 Confirming the applicability of J1 and H2 riboswitches downstream of T7 promoter using GFPuv as a reporter protein. The experiment was done using the invitro PUREfrex TX-TL system (Gene Frontier) as usual.

FIG. 14 Testing J1 and H2 riboswitches downstream of pTac inside Linear E. coli S30 Extract System (Promega) using mCherry as a reporter protein.

### DESCRIPTION OF THE EMBODIMENTS

The present inventors have carried out dedicated research to solve the above-mentioned problem, thereby a nucleotide sequence having a specific binding ability to histidine has been specified. Specifically, regarding the aptamer, we successfully selected an RNA aptamer that can bind histamine with high affinity and specificity from a pool of 3×10¹⁴ random sequences through SELEX aided by deep sequencing. Minimization of the initially selected aptamer through reselection and affinity measurements resulted in a 37-nt aptamer (A1-949) of which 25 nucleotides appear to be responsible for histamine recognition. To demonstrate a practical application of the aptamer, we engineered a fluorescence based aptasensor that can detect histamine concentration as low as 1 μM.

Furthermore, through both rational design and experimentation, we employed the aptamer mentioned above to develop histamine-responsive synthetic riboswitch using in-vitro transcription/translation system (PURE system).

### Aptamer

The nucleic acid aptamer of the present invention is, as mentioned above, a nucleic acid aptamer that binds to histamine, comprising the base sequence (i) or (ii) below:

(i) the base sequence of SEQ ID NO: 1;

(ii) the base sequence comprising substitution(s), deletion(s), and/or addition(s) of 1 to 3 base(s) in the base sequence of SEQ ID NO: 1.

(SEQ ID NO: 1)
5′-CCAGUGGGUUGAAGGAAAGUAACAG-3′

Here, in the above base sequence, “U” means “U/T (U or T)”, that is, when the base sequence is DNA base sequence, U (uracil) is replaced by T (thymine) (the same applies hereinafter).

In the present invention, a “nucleic acid aptamer” is, for example, a nucleic acid molecule having a short sequence of approximately 20 to 80 base length and it refers to a single-stranded nucleic acid molecule comprising the polynucleotide that has the specific base sequence and can specifically recognize a molecule and substance that would be a target. The nucleic acid aptamer of the present invention is a single-stranded nucleic acid molecule (a single-stranded polynucleotide) having a function specifically binding to histamine. Note that in the present specification, the base sequence is described from the left to the right, from 5′ end to 3′ end. Moreover, when the aptamer is a DNA, U is replaced by T in the base sequence.

Here, the term “specific” or “specifically ” in the present specification refers to a selective binding of a nucleic acid aptamer of the present invention to histamine. The binding specificity of an aptamer can be examined by comparing the binding of the aptamer to histamine (the binding force to histamine) to the binding of the aptamer to an irrelevant substance (the binding force to an irrelevant substance), under a predetermined condition. The nucleic acid aptamer of the present invention is considered as being specific, when the binding force to histamine is at least twice, at least five times, at least seven times, and preferably at least ten times than the binding force to an irrelevant substance. For example, in the nucleic acid aptamer of the present invention, the binding force to histamine is higher than the binding force to histidine.

The binding force of the nucleic acid aptamer to a substance is, for example, represented by a dissociation constant (Kd) of the nucleic acid aptamer with the substance. The dissociation constant of the nucleic acid aptamer of the present invention for histamine is, for example, no more than 5 μM, preferably no more than 1 μM, and more preferably no more than 0.1 μM. In contrast, the dissociation constant of the nucleic acid aptamer of the present invention for histidine is, for example, no less than than 40 μM, preferably no less than 100 μM and more preferably no less than 500 μM.

The nucleic acid aptamer of the present invention may comprise (i) the base sequence of SEQ ID NO: 1, and may comprise (ii) the base sequence comprising substitution(s), deletion(s), and/or addition(s) of 1 to 3 base(s) in the base sequence of SEQ ID NO: 1 as long as the nucleic aptamer has the function to specifically bind to histamine. Moreover, the number of substitution(s), deletion(s), and addition(s) of the base sequence is preferably 1 to 2 nucleotide(s), more preferably 1 nucleotide.

Preferably, in the nucleic acid aptamer of the present invention, when such substitution(s) of the base sequence is present, the substitution(s) is/are made to at least one position of position 5 (U), position 16 (A), position 17 (A) and position 18 (A) of SEQ ID NO: 1.

In the nucleic acid aptamer of the present invention, the nucleic acid (nucleotide) which the aptamer is built from is at least one selected from the group consisting of DNA (D-form), RNA (D-form), and artificial nucleic acid. Examples of the artificial nucleic acid include L-DNA and L-RNA. Preferably, the nucleic acid is DNA (D-form), RNA (D-form), L-DNA or L-RNA, more preferably L-DNA or L-RNA, yet more preferably L-RNA.

The number of bases in a polynucleotide which the nucleic acid aptamer of the present invention consists of may be up to 60 bases, preferably up to 40 bases, and more preferably up to 35 bases.

Preferably, in the nucleic acid aptamer of the present invention, the nucleic acid aptamer has a stem-loop structure. In order to form a stem-loop structure, base sequences that can form a double-stranded stem structure are added to both ends of the base sequence (i) or (ii). Due to the fact that the aptamer forms a double-stranded stem structure, a loop structure can be easily formed and an ability to bind to histamine is improved. Here, usually, in the stem-loop structure, the base sequence of the loop structure make a significant contribution to binding specificity, the base sequence of the stem structure is less important.

In the present invention, the double-stranded stem structure may be formed by bonding of two or more places of a nucleic acid aptamer to each other. For example, the stem structure may be a structure formed by base pairing of one or more sets of complementary bases. The means of bonding for forming a stem structure is not limited to a means by the formation of base pairs and may be a means by other arbitrary cross-linked structures.

In the case where the double-stranded stem structure is formed by base pairing, for example, the first base sequence which is the complementary base sequence of the second base sequence is added to the 5′ end of the base sequence (i) (or (ii)), the second base sequence is added to the 3′ end of the base sequence (i) (or (ii)). Then the first base sequence and the second base sequence can form a double-stranded stem structure by complementary base pairing. Here, the base number of one base sequence forming the double-stranded stem structure may be 2 to 10 bases, preferably 3 to 8 bases, and more preferably 4 to 6 bases.

Preferably, stem base sequence 1/stem base sequence 2 is added to the 5′/3′ end of the base sequence (i) or (ii). Specifically, stem base sequence 1 is added to the 5′ end or the 3′ end of the base sequence (i) (or (ii)), stem base sequence 2 is added to the other end of the base sequence (i) (or (ii)).

5′-UACGAU-3′ (stem base sequence 1)
5′-AUCGUA-3′ (stem base sequence 2)

More preferably, the nucleic acid aptamer of the present invention comprises the base sequence of SEQ ID NO: 7. or 8.

(SEQ ID NO: 7)
5′-UACGAUCCAGUGGGUUGAAGGAAAGUAACAGAUCGUA-3′
(SEQ ID NO: 8)
5′-AUCGUACCAGUGGGUUGAAGGAAAGUAACAGUACGAU-3′

Furthermore, for example, the nucleic acid aptamer of the present invention can have a labeling substance as a detection label in order to detect histamine as described hereinafter. The labeling substance may be bound to the 5′ end or 3′ end. As such a detection label, fluorescent label is preferred; however, Raman label, enzyme label, and infrared label may be used. A labelling substance can be appropriately selected depending on the type of detection labels.

Examples of the labeling substance includes Cy3, Cy5, Cy7, fluorescein, Texas Red, and TAMRA.

Moreover, when using a fluorescent label as the detection label, a quencher substance that absorbs a fluorescence energy emitted from the fluorescent substance (fluorescent label) may be used together. In such embodiment, fluorescence which occurs when the separation of the fluorescent substance and the quencher substance at the time of detection reaction is detected.

The nucleic acid aptamer of the present invention is typically a polynucleotide, thus, it can be linked to other polynucleotide.

### Composition, Kit and Biosensor

The composition for histamine detection of the present invention comprises the nucleic acid aptamer of the present invention. The histamine detecting kit of the present invention comprises the aptamer of the present invention. The biosensor (aptasensor) for histamine detection of the present invention comprises the nucleic acid aptamer of the present invention.

The composition, kit and biosensor of the present invention comprises the nucleic acid aptamer of the present invention. The nucleic acid aptamer of the present invention can be used for the detection of histamine as mentioned above, therefore, the composition, kit and biosensor of the present invention can be used for the detection of histamine.

The composition, kit and biosensor of the present invention may further comprise any components other than the nucleic acid aptamer of the present invention, if necessary.

### Method For Detecting Histamine

The method for detecting histamine of the present invention is characterized by using the nucleic acid aptamer of the present invention. The method of the present invention can detect histamine in a subject sample.

The method for detecting histamine of the present invention comprises, for example, the following steps. First, a complex including the nucleic acid aptamer of the present invention and a nucleic acid fragment is prepared. The nucleic acid fragment has a base sequence complementary to a part of the nucleic acid aptamer, and can bind to the part of the nucleic acid aptamer. In the complex, the nucleic acid aptamer forms a double strand with the nucleic acid fragment.

Next, the nucleic acid fragment is separated from the nucleic acid aptamer by binding histamine in subject sample with the nucleic acid aptamer. That is, when the structure of the nucleic acid aptamer is changed by the binding the nucleic acid aptamer to histamine, the cleavage (separation) of the double strand occurs. This structure change occurs when a bond between the nucleic acid aptamer and histamine is stronger than a bond between the nucleic acid aptamer and the nucleic acid fragment. When the double stranded in the complex is cleaved in this way, the nucleic acid fragment is dissociated from the nucleic acid aptamer.

Next, the cleavage of the double strand in the complex is detected. In this case, the detection of the cleavage of the double strand in the nucleic acid aptamer is not limited as long as physical and chemical changes caused by the cleavage of the double strand in the complex can be detected. For example, the detection refers to the detection of changes in signals such as optical signals, electrical signals, and color signals.

In the method of the present invention, the molar ratio of the nucleic acid fragment to the nucleic acid aptamer may be from 2:1 to 1000:1, preferably from 5:1 to 500:1, and more preferably from 10:1 to 100:1.

In the method of the present invention, the nucleic acid aptamer and the nucleic acid fragment may be DNA, RNA, or artificial nucleic acid independently. In addition, the nucleic acid aptamer in the method of the present invention may be bound to other polynucleotide.

In the method of the present invention, the length of the base sequence of the nucleic acid aptamer may be from 12 to 80, preferably from 15 to 50, and more preferably from 20 to 35. The length of the base sequence of the nucleic acid fragment may be up to 20, preferably up to 15, and more preferably up to 7. Preferably, when the length of the base sequence of the nucleic acid aptamer is 37 bases, the length of the base sequence of the nucleic acid fragment is 13 bases.

In the method of the present invention, each of the steps is typically performed in an assay solution. Here the assay solution is, for example, a buffer solution such as HEPES. The assay solution contains any components such as NaCl and MgCl₂. The NaCl concentration may be 0 to 1000 mM, preferably 10 mM to 500 mM, and more preferably 50 mM to 250 mM. The MgCl₂ concentration may be 0 mM to 50 mM, preferably 0 mM to 10 mM, and more preferably 0 mM to 3 mM.

In order to easily detect the cleavage of the double strand in the complex, a fluorescent substance may be bound to the nucleic acid aptamer and a quencher substance may be bound to the nucleic acid fragment. Here the fluorescent substance and the quencher may be bound to any positon as long as a quencher substance that absorbs a fluorescence energy emitted from the fluorescent substance. In addition, a quencher substance may be bound to the nucleic acid aptamer and a fluorescent substance may be bound to the nucleic acid fragment.

Examples of the fluorescent substance and the quencher substance used in the method includes fluorescein, Cy5, Cy3, TET, ROX, TAMRA (fluorescent substance) and DABCYL, BHQ-1, BHQ-2, BHQ-3 (quencher substance).

### Riboswitch

The riboswitch of the present invention comprises the nucleic acid aptamer of the present invention. Riboswitch typically refers to a functional unit (region or segment) of a mRNA molecule for regulating the expression of proteins encoded by the mRNA molecule. Typically, the riboswitch of the present invention is a RNA polynucleotide and comprises the nucleic acid aptamer of the present invention in a part thereof. Since the nucleic acid aptamer of the present invention undergoes a structural change by binding to histamine as described above, the structure of the riboswitch itself is changed under the influence of the structural change of the nucleic acid aptamer of the present invention. Accordingly, the riboswitch of the present invention refers to a RNA molecule (region or segment) which detects the presence of histamine, changes its structures, and regulates the expression of protein coded by the mRNA molecule which the riboswitch connects to (or which the riboswitch is included in).

The riboswitch of the present invention may comprises any base sequence as long as it comprises the base sequence of the nucleic acid aptamer of the present invention and can detect the presence of histamine and regulate the expression of protein coded by the mRNA which the riboswitch connects to (or which the riboswitch is included in). Moreover, the base sequence may include a part of a coding region for the protein whose expression is to be regulated by the riboswitch. In the present invention, when the riboswitch detects histamine, gene expression is usually turned on, but may be turned off.

For example, the riboswitch of the present invention may comprises any of the base sequences of SEQ ID NOs: 2 to 6.

(SEQ ID NO: 2)
5′-GAAGGAUCCAGUGGGUUGAAGGAAAGUAACAGAUCCUCCAGGAUGC
GACCCGGUUAUCGAAUUAAGGAGGUAAAAAAUGCAAGUCGACCUGCUGG
AUCCAAAC-3′
(SEQ ID NO: 3)
5′-GAAGGAUCCAGUGGGUUGAAGGAAAGUAACAGAUCCUUUAGGAUGC
GACCCGGUUAUCGAAUUAAGGAGGUAAAAAAUGCAAGUCGACCUGCUGG
AUCCAAAC-3′
(SEQ ID NO: 4)
5′-GAAGGAUCCAGUGGGUUGAAGGAAAGUAACAGAUCCUCCAGGAUGC
GACCCGGUUAUCGAAUUAAGGAGGUAAAAAAUGCAAGUCGACCUGCUCG
AUCCAAAC-3′
(SEQ ID NO: 5)
5′-GAGGAUCCAGUGGGUUGAAGGAAAGUAACAGAUCCUUCAGGAUGCG
ACCCGGUUAUCGAAUUAAGGAGGUAAAAAAUGCAAGUCGACCUGCUGGA
UCCAAAC-3′
(SEQ ID NO: 6)
5′-GACGAUCCAGUGGGUUGAAGGAAAGUAACAGAUCGUUCAGGAUGCG
ACGCGCUUGGAAUUAAGGAGGUAAAAAAUGCAAGUCGACCUGCUGGAUC
CAAAC-3′

### RNA Polynucleotide

The RNA polynucleotide of the present invention comprises a regulation region corresponding to the riboswitch of the present invention and a cording region for a protein. The regulation region is arranged in the RNA polynucleotide so that the expression of the protein encoded by the coding region can be regulated by the regulation region. Here the regulation region is the riboswitch of the present invention, thus, when the RNA polynucleotide contact with histamine, the expression of the protein is turned on or turned off. In the present invention, the regulation region and the coding region may overlap.

### Nucleic Acid Construct

The nucleic acid construct of the present invention is a nucleic acid molecule comprising a promoter sequence and a base sequence complementary to the RNA polynucleotide of the present invention. Here the base sequence complementary to the RNA polynucleotide of the present invention is operationally linked to the promoter sequence such that when the nucleic acid construct is introduced into an appropriate host cell, said RNA polynucleotide will be transcribed in a normal condition in the host cell.

### EXAMPLES

### 1. Materials and Methods

### 1.1. Oligonucleotides and Molecular Biology Reagents

A DNA library with 40 degenerate bases flanked by 23-base constant sequences (tempN40) and the primers (Reverse selection primer, and T7-promoter forward primer) for reverse transcription and PCR during SELEX were purchased from Trilink Biotechnologies (catalog# 0-32001-40) (Table 1). Partially randomized DNA based on the A1 aptamer sequence (temp-A1-doped) was synthesized by Gene Design and contained 79% original (A1 aptamer) base mixed with 7% each of the remaining three bases (Table 1). OneTaq(registered trademark) 2× Master Mix (NEB) and Q5 High-Fidelity 2× Master Mix (NEB) were used for PCR in SELEX and sequencing library preparation, respectively. Reverse transcription reactions were performed using Maxima H Minus Reverse Transcriptase (Thermo Scientific). HiScribe T7 Quick High Yield RNA Synthesis Kit (NEB) was used for in vitro transcription of the RNA pool for the first SELEX round, and T7 RNA polymerase (NEB) was used for the subsequent rounds. The minimized RNA aptamer labeled with Cy5 at the 5′ end (Cy5-A1-949) and 3′ BHQ2-labeled DNAs (QueN: N=8, 10-15, 17) were purchased from FASMAC and Macrogen, respectively (Table 1). The 5′-Cy3-labeled RNA aptamer (Cy3-A1-949*) and 3′ BHQ2-labeled DNA (Que13*) in L-enantiomer forms were synthesized by Gene Design.

TABLE 1
Oligonucleotides used in this study

| Name | Sequence |
|---|---|
| N40 DNA library (tempN40) | 5′ TAGGGAAGAGAAGGACATATGAT(N40)TTGACTAGTACATGACCACTTGA 3′ |
| | (SEQ ID NO: 11) |
| Reverse selection primer | 5′ TCAAGTGGTCATGTACTAGTCAA 3′ (SEQ ID NO: 12) |
| T7-promoter forward Primer | 5′ TTCAGGTAATACGACTCACTATAGGGAAGAGAAGGACATATGAT 3′ (SEQ ID |
| | NO: 13) |
| temp-A1-doped | 5′ TAGGGAAGAGAAGGACATATGATCCAGTGGGTTGAAGGAAAGTAACAGATCG |
| | TATGAGCGGGGTTGACTAGTACATGACCACTTGA 3′ (SEQ ID NO: 14) |
| Cy5-A1-949 | 5′ Cy5-UACGAUCCAGUGGGUUGAAGGAAAGUAACAGAUCGUA 3′ (SEQ ID |
| | NO: 15) |
| Que 8 | 5′ GGATCGTA-BHQ2 3′ |
| Que 10 | 5′ CTGGATCGTA-BHQ2 3′ (SEQ ID NO: 16) |
| Que 11 | 5′ ACTGGATCGTA-BHQ2 3′ (SEQ ID NO: 17) |
| Que 12 | 5′ CACTGGATCGTA-BHQ2 3′ (SEQ ID NO: 18) |
| Que 13 | 5′ CCACTGGATCGTA-BHQ2 3′ (SEQ ID NO: 19) |
| Que 14 | 5′ CCCACTGGATCGTA-BHQ2 3′ (SEQ ID NO: 20) |
| Que 15 | 5′ ACCCACTGGATCGTA-BHQ2 3′ (SEQ ID NO: 21) |
| Que 17 | 5′ CAACCCACTGGATCGTA-BHQ2 3′ (SEQ ID NO: 22) |

N indicates A, G, C, or T. Underlined sequence is the doped part of the A1 aptamer

### 1.2. Immobilization of Histidine on Agarose Beads

EAH Sepharose 4B (GE Healthcare) was employed as an immobilization matrix in PD-10 columns (GE Healthcare). To enable the immobilization of histamine without masking its free primary amine and imidazole groups, L-histidine was immobilized instead through its carboxyl group. For that, Fmoc-L-histidine (Watanabe Chemicals, Japan) was coupled to the matrix as previously described (Illangasekare and Yarus 2002). Ten milliliters of EAH Sepharose 4B (GE Healthcare) matrix was transferred to a PD-10 (GE Healthcare) column and washed twice with 5 ml dimethylformamide (DMF) (Nacalai). The matrix was suspended in 5 ml DMF containing 30 μmol Fmoc-L-histidine (Watanabe Chemical Industries) and 30 μmol benzotriazol-1-yl-oxytripyrrolidinophosphonium hexafluorophosphate (PyBOP) (Watanabe Chemicals). A total of 500 μmol of diisopropylethlamine (DIPEA) (Nacalai) was slowly added while stirring, and the reaction was allowed to proceed for 2 h at 24 ° C. After washing the matrix with DMF (2×5 ml), it was incubated for 30 min with 5 ml DMF containing 2 mmol acetic anhydride (Nacalai) and 2 mmol DIPEA to block the unreacted amino groups on the beads. After washing the matrix with DMF (4×5 ml), it was treated twice with 5 ml of 20% piperidine (Nacalai) in DMF for 10 min to remove the Fmoc group. The eluents from the two piperidine treatments were combined and the absorbance of the solution at 301 nm (ε_{M}=9700) (Illangasekare and Yarus 2002) was measured using 20% piperidine in DMF as a blank to determine the amount of the released Fmoc to calculate the coupling efficiency. It was determined that 14.2 μmol of histidine was coupled which corresponds to approximately 19% of the amino groups on the matrix. The counterselection matrix was prepared in parallel using the same method except in the absence of histidine.

### 1.3. SELEX Procedure

The DNA library template tempN40 (0.5 nmol, 3×10¹⁴ molecules) and the reverse selection primer (1 nmol) were mixed in a total volume of 1.5 ml using OneTaq (registered trademark) 2X Master Mix (NEB). The mixture was denatured at 9420 C. for 4 min followed by four cycles of 9420 C., 4820 C., and 6820 C. for 30 s each. Subsequently, 2 nmol of T7-promoter forward primer were added and four additional thermal cycles were performed to generate the template for in vitro transcription. The template DNA was column purified (DNA Clean & Concentrator™-5, Zymo Research) and 20 μg were used for an overnight RNA transcription reaction in 270 μl volume using HiScribe T7 High Yield RNA Synthesis Kit. 250 μl of the RNA solution were then added to 125 μl of 10×0 DNase I buffer and treated by DNase I (NEB) in 1250 μl reaction for 1 h. The RNA was then purified through ethanol precipitation.

The random RNA pool (110 μg corresponding to approximately 2.3×10¹⁵ molecules) was dissolved in 2.5 ml of the selection buffer (HEPES 50mM, pH 7.0, NaCl 250 mM, MgCl₂ 5 mM, CaCl₂ 5 mM, and glycine 2 mM) (Majerfeld et al. 2005), heated to 7020 C. for 10 min (before adding the MgCl₂ and CaCl₂ salts), and slowly cooled to room temperature. The RNA library was then incubated with 500 μl of the histidine-coupled matrix in the selection column for 1 h at 3720 C. The unbound RNA molecules were then washed once using 0.5 ml of the selection buffer, and the bound sequences were eluted using 2×1 ml of the selection buffer containing 10 mM histamine. The eluted RNA molecules were then ethanol precipitated after the addition of Quick-Precip Plus Solution (EdgeBio), reverse transcribed, PCR amplified, and in vitro transcribed to produce the RNA pool for the second round. Starting from the second round, 1.5 μg of RNA dissolved in 2 ml selection buffer was applied to the matrix, and each cycle was preceded with a counterselection in 500 μl of the acetylated matrix. Starting from the fourth round, mutagenic PCR was employed to increase the diversity of the library (Wilson and Keefe 2001). A negative selection step using L-histidine was also included from round 8. Histamine concentration during the elution step was reduced to 0.2 mM in rounds 9-11. Other selection conditions for each round are summarized in Table 2.

TABLE 2
Detailed conditions for each round in the SELEX process.

| | | | | | | |
|---|---|---|---|---|---|---|
| | | | No. of | No. of | L-histidine | |
| | CaCl₂ | MgCl₂ | washings | negative | Concentration | histamine |
| | concentration | concentration | using the | selections | in negative | Concentration |
| | in the buffer | in the buffer | buffer | using L- | selection | in the eluent |
| Round | (mM) | (mM) | only | histidine | solution (mM) | (mM) |

1^{st} SELEX process

| | | | | | | |
|---|---|---|---|---|---|---|
| Round 1 | 5 | 5 | 1 × 0.5 ml | zero | zero | 10 |
| Round 2 | 5 | 5 | 2 × 0.5 ml | zero | zero | 10 |
| Round 3 | 5 | 5 | 3 × 0.5 ml | zero | zero | 10 |
| Round 4 | zero | 1 | 3 × 0.5 ml | zero | zero | 10 |
| Round 5 | zero | 1 | 3 × 0.5 ml | zero | zero | 10 |
| Round 6 | zero | 1 | 3 × 0.5 ml | zero | zero | 10 |
| Round 7 | zero | 1 | 3 × 0.5 ml | zero | zero | 10 |
| Round 8 | zero | 0.1 | 3 × 0.5 ml | 1 × 1 ml | 0.2 | 10 |
| Round 9 | zero | 0.1 | 3 × 0.5 ml | 2 × 1 ml | 0.5 | 0.2 |
| Round 10 | zero | 0.1 | 3 × 0.5 ml | 2 × 1 ml | 0.5 | 0.2 |
| Round 11 | zero | 0.1 | 3 × 0.5 ml | 3 × 1 ml | 0.5 | 0.2 |

Three columns were used in rounds 8, 9, and 11 for SELEX and to prepare sequencing samples. One column was used for regular SELEX with a negative selection using histidine and eluted with histamine. The resulting RNAs were used for the subsequent round of SELEX. The second column was used to perform the same SELEX procedure, but the bound RNA was eluted with histidine (0.2 mM) after the buffer wash step. The third column was similarly eluted with histamine (0.2 mM) after the buffer wash without a negative selection step using histidine. The latter two eluents and the RNA pool before selection from these rounds were separately reverse transcribed and PCR amplified to attach barcodes adapter sequences for deep sequencing. The sequencing samples were analyzed by Illumina MiSeq DNA sequencer using MiSeq reagent kit v3. Sequences with more than 10 reads in the histamine elution from round 11 were compiled.

### 1.4. Reselection of Partially Randomized A1 Aptamer

A doped RNA library based on the A1 aptamer (SEQ ID NO: 9) selected from the random RNA pool was prepared using temp-A1-doped as described above. The selection buffer contained HEPES (50 mM, pH 7.0), NaCl (250 mM), MgCl₂ (0.1 mM), and glycine (2 mM). The procedure was similar to the first selection with the following exceptions. The first round was performed using 100 μg RNA dissolved in 6 ml and the subsequent rounds were performed using 10 μg RNA in 2 ml. More stringent histidine negative selections were performed, starting with 1 mM in the first round and 50 mM in the fourth round. Detailed selection conditions are summarized in Table 3. The initial RNA library before SELEX and the eluted RNA pools after each round were analyzed by deep sequencing as described above. Sequences with more than five reads in the final round of the selection were compiled.

TABLE 3
Detailed conditions for each round in the SELEX process.

| | | | | | | |
|---|---|---|---|---|---|---|
| | | | No. of | No. of | L-histidine | |
| | CaCl₂ | MgCl₂ | washings | negative | Concentration | histamine |
| | concentration | concentration | using the | selections | in negative | Concentration |
| | in the buffer | in the buffer | buffer | using L- | selection | in the eluent |
| Round | (mM) | (mM) | only | histidine | solution (mM) | (mM) |

2^{nd} SELEX process

| | | | | | | |
|---|---|---|---|---|---|---|
| Round 1 | zero | 0.1 | 3 × 0.5 ml | 3 × 1 ml | 1 | 0.2 |
| Round 2 | zero | 0.1 | 3 × 0.5 ml | 3 × 1 ml | 5 | 0.2 |
| Round 3 | zero | 0.1 | 3 × 0.5 ml | 3 × 1 ml | 25 | 0.2 |
| Round 4 | zero | 0.1 | 3 × 0.5 ml | 3 × 1 ml | 50 | 0.1 |

### 1.5. Isothermal Titration Calorimetry (ITC)

Both A1 aptamer (SEQ ID NO: 9) and the minimized A1-949 aptamer (SEQ ID NO: 10) were in vitro transcribed using the HiScribe T7 Quick High Yield RNA Synthesis Kit (NEB) according to the manufacturer's instructions and DNase I treated. The reaction solutions were extracted with phenol/chloroform extraction and RNAs were precipitated by ethanol. The aptamers were dissolved in the buffer used in the SELEX selection as described above and purified using Amicon Ultra 4 mL Centrifugal Filters (Ultracel) with a 3 kDa cutoff to remove remaining truncated transcripts and nucleotides. MicroCal PEAQ-ITC calorimeter (Malvern) was used. The RNA was added in the cell while the ligand (histamine or L-histidine) was titrated from the syringe. The cell temperature was maintained at 2520 C. Control reactions in which the ligand was titrated against the buffer, the buffer was titrated against the RNA solution, and the buffer was titrated against the buffer were also performed and used to correct the baseline.

A1 aptamer:
(SEQ ID NO: 9)
5′-UAGGGAAGAGAAGGACAUAUGAUCCAGUGGGUUGAAGGAAAGUAAC
AGAUCGUAUGAGCGGGGUUGACUAGUACAUGACCACUUGA-3′
A1-949 aptamer:
(SEQ ID NO: 10)
5′-UACGAUCCAGUGGGUUGAAGGAAAGUAACAGAUCGUA-3′

### 1.6. Microscale Thermophoresis

Cy5-A1-949 was used for the assay. The binding for both L-histidine and histamine was measured using the same buffer used for the SELEX at 2520 C. The experiment was performed by 2bind GmbH.

### 1.7. Aptasensor Based Assay of Histamine

Both the fluorescently labeled aptamer Cy5-A1-949 and the quencher DNA QueN were stored as 100 μM stocks at -8020 C. Unless otherwise indicated, Cy5-A1-949 was diluted to 11 nM and QueN was diluted to 44 nM in the assay buffer. The composition of the optimized buffer used for the assay was HEPES (50mM, pH 7.0) with NaCl (125 mM). Aliquots (90 μl) of Cy5-A1-949 and QueN solution were dispensed in the wells of a black 384-well microplate (Thermo Scientific) and the samples (10 μl) prepared in the same buffer were added to each well. The plate was incubated at 6520 C. for 20 min and then cooled at room temperature for 1 h. Subsequently, fluorescence of the samples was measured by exciting at 650 nm and observing the emission at 670 nm using M1000PRO microplate reader (Tecan). Measurements using the L-enantiomer sensor oligonucleotides Cy3-A1-949* and Que13* were performed with 550 nm excitation and 570 nm emission.

### 1.8. Measurement of Spiked Histamine in Tuna Samples Using the Aptasensor

Fresh skinless sliced tuna fish muscle pieces from the dorsal part were purchased at a local market and stored at −2020 C. until use. Approximately 1.5 to 2 g pieces were cut and added to 5 ml Eppendorf tubes. An equivalent volume of the assay buffer spiked with the indicated concentration of histamine was then added (100 μl for every 100 mg of tuna) to each tube. The mixture was vortexed, homogenized, and heated to 9020 C. for 15 min. The sample was centrifuged and the supernatant was collected. The supernatant (400 μl) was filtered through 3 kDa Molecular weight cutoff filter (Amicon (registered trademark) Ultra 0.5 mL Centrifugal Filters (Ultracel (registered trademark))), and the filtrate was used for the assay as described above.

### 1.9. Riboswitch Design and Testing Using PURE In-Vitro TX-TL System

DNA sequence encoding mCherry protein was PCR amplified from plasmid Psup-1753-mCherry (Dwidar and Yokobayashi 2017) and used as a reporter protein under control of T7 promoter. We initially employed an Optimum RBS sequence for expressing mCherry which was obtained using RBS calculator tool. A1-949 aptamer was then inserted between the T7 promoter and this RBS sequence. Subsequently, the riboswitches were constructed using a series of rational design with the aid of the online Vienna RNA folding tool and in-vitro testing. In-vitro experiments were done using the PUREFREX in-vitro TX-TL system. Briefly, the DNA for the tested riboswitch was added at a final concentration of 12 ng/μl in a total of 10 μl reaction which was done in absence or presence of histamine at the indicated concentration. The reaction was incubated for 4 h at 3720 C. inside a thermocycler then 8 μl were transferred to white low-volume 384-well plate (Corning, C. N. 5413). mCherry fluorescence (587 nm/610 nm) was then measured in Tecan M1000PRO microplate reader. The successful riboswitch constructs were then cloned into plasmids, transformed into E. coli TOP10 and their sequences were verified before proceeding to further experiments. The sequences of the selected successful riboswitches are shown below (Tables 4-12).

Sequences of the riboswitches designed in this study.

TABLE 4
pT7-Optimum RBS-mCherry

TAATACGACTCACTATAGACTCACATACGAGATATATAGAAGGAGGTAATAAATGGCAATC
ATTAAGGAGTTCATGAGATTCAAAGTTCACATGGAAGGTTCTGTAAATGGACATGAATTTGA
AATAGAAGGTGAAGGAGAAGGAAGGCCTTATGAAGGAACCCAAACCGCGAAGCTAAAAGTTA
CTAAGGGTGGCCCATTACCATTTGCATGGGATATCCTTAGCCCTCAATTCATGTATGGGTCAA
AGGCTTATGTCAAGCACCCCGCCGACATTCCAGACTATCTAAGTTATCTTTTCCCGAAGGGT
TTAAGTGGGAGCGTGTGATGAACTTCGAAGACGGTGGCGTGGTAACAGTGACTCAGGATTCG
TCCCTGCAAGATGGTGAATTTATCTACAAAGTCAAATTAAGAGGAACTAACTTTCCATCTGAC
GGCCCGGTTATGCAAAAAAAGACAATGGGCTGGGAGGCCTCCTCAGAACGAATGTACCCTGA
AGATGGTGCCTTGAAGGGTGAGATTAAACAAAGATTGAAATTGAAAGATGGTGGACATTATGA
CGCTGAGGTTAAAACGACATACAAAGCTAAGAAACCTGTCCAGCTCCCAGGTGCTTACAATG
TAAATATAAAACTTGATATTACATCACATAATGAAGATTATACGATAGTTGAACAATACGAAAG
GGCTGAGGGGAGACATAGTACTGGTGGCATGGATGAACTATACAAAGGTTCTGGTACCGCAT
AACTAGCATAACCCCTCTCTAAACGGAGGGGTTTGAATTCTAT (SEQ ID NO: 23)
Note:
17 bases from the 5′ terminal end indicate T7 promoter sequence. Bold letters indicate the sequences constituting the Optimum RBS and the leader peptide sequence fused to mCherry. Gray letters indicate the coding sequence of mCherry. The underlined G indicates the transcription starting position, and the underlined ATG indicates the translation starting position.

TABLE 5
pT7-H2 riboswitch -mCherry

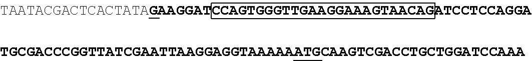

CATGAGATTCAAAGTTCACATGGAAGGTTCTGTAAATGGACATGAATTTGAAATAGAAGGTGA
AGGAGAAGGAAGGCCTTATGAAGGAACCCAAACCGCGAAGCTAAAAGTTACTAAGGGTGGCC
CATTACCATTTGCATGGGATATCCTTAGCCCTCAATTCATGTATGGGTCAAAGGCTTATGTCA
AGCACCCCGCCGACATTCCAGACTATCTAAAGTTATCTTTTCCCGAAGGGTTTAAGTGGGAG
CGTGTGATGAACTTCGAAGACGGTGGCGTGGTAACAGTGACTCAGGATTCGTCCCTGCAAGA
TGGTGAATTTATCTACAAAGTCAAATTAAGAGGAACTAACTTTCCATCTGACGGCCCGGTTAT
GCAAAAAAAGACAATGGGCTGGGAGGCCTCCTCAGAACGAATGTACCCTGAACATGGTGCCT
TGAAGGGTGAGATTAAACAAAGATTGAAATTGAAAGATGGTGGACATTATGACGCTGAGGTTA
AAACGACATACAAAGCTAAGAAACCTGTCCAGCTCCCAGGTGCTTACAATGTAAATATAAAAC
TTGATATTACATCACATAATGAAGATTATACGATAGTTGAACAATACGAAAGGGCTGAGGGGA
GACATAGTACTGGTGGCATGGATGAACTATACAAAGGTTCTGGTACCGCATAACTAGCATAAC
CCCTCTCTAAACGGAGGGGTTTGAATTCTAT (SEQ ID NO: 24)
Note:
17 bases from the 5’ terminal end indicate T7 promoter sequence. Bold letters indicate the sequences constituting the riboswitch. Gray letters indicate the coding sequence of mCherry. The underlined G indicates the transcription starting position, and the underlined ATG indicates the translation starting position. The box indicates the base sequence of SEQ ID NO: 1.

For the other riboswitches, the bold letters part of the sequence (riboswitch part) in pT7-H2 riboswitch -mCherry was changed as follows (Tables 6-9):

TABLE 6
J1 riboswitch

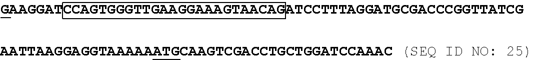

Note:
Bold letters indicate the sequences constituting the riboswitch. The underlined G indicates the transcription starting position, and the underlined ATG indicates the translation starting position. The box indicates the base sequence of SEQ ID NO: 1.

TABLE 7
W2 riboswitch

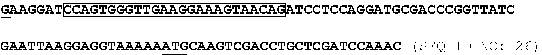

Note:
Bold letters indicate the sequences constituting the riboswitch. The underlined G indicates the transcription starting position, and the underlined ATG indicates the translation starting position. The box indicates the base sequence of SEQ ID NO: 1.

H1 riboswitch

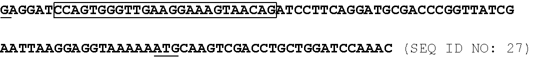

Note:
Bold letters indicate the sequences constituting the riboswitch. The underlined G indicates the transcription starting position, and the underlined ATG indicates the translation starting position. The box indicates the base sequence of SEQ ID NO: 1.

TABLE 9
C2 riboswitch

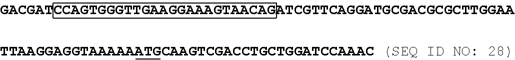

Note:
Bold letters indicate the sequences constituting the riboswitch. The underlined G indicates the transcription starting position, and the underlined ATG indicates the translation starting position. The box indicates the base sequence of SEQ ID NO: 1.

TABLE 10
pT7-H2 riboswitch -GFPuv

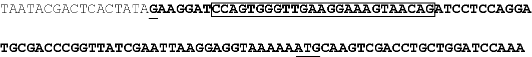

CATGAGTAAAGGAGAAGAACTTTTCACTGGAGTTGTCCCAATTCTTGTTGAATTAGATGGTGA
TGTTAATGGGCACAAATTTTCTGTCAGTGGAGAGGGTGAAGGTGATGCAACATACGGAAAAC
TTACCCTTAAATTTATTTGCACTACTGGAAAACTACCTGTTCCATGGCCAACACTTGTCACTA
CTTTCTCTTATGGTGTTCAATGCTTTTCCCGTTATCCGGATCATATGAAACGGCATGACTTTT
TCAAGAGTGCCATGCCCGAAGGTTATGTACAGGAACGCACTATATCTTTCAAAGATGACGGG
AACTACAAGACGCGTGCTGAAGTCAAGTTTGAAGGTGATACCCTTGITAATCGTATCGAGTTA
AAAGGTATTGATTTTAAAGAAGATGGAAACATTCTCGGAGACAAACTCGAGTACAACTATAAC
TCACACAATGTATACATCACGGCAGACAAACAAAAGAATGGAATCAAAGCTAACTTCAAAATT
CGCCACAACATTGAAGATGGATCCGTTCAACTAGCAGACCATTATCAACAAAATACTCCAATT
GGCGATGGCCCTGTCCTTTTACCAGACAACCATTACCTGTCGACACAATCTGCCCTTTCGAA
AGATCCCAACGAAAAGCGTGACCACATGGTCCTTCTTGAGTTTGTAACTGCTGCTGGGATTA
CACATGGCATGGATGAGCTCTACAAATAACTAGCATAACCCCTCTCTAAACGGAGGGGTTTGA
ATTCTAT (SEQ ID NO: 29)
Note:
bases from the 5’ terminal end indicate T7 promoter sequence. Bold letters indicate the sequences constituting the riboswitch. Gray letters indicate the coding sequence of GFP. The underlined G indicates the transcription starting position, and the underlined ATG indicates the translation starting position. The box indicates the base sequence of SEQ ID NO: 1.

TABLE 11
pTac -H2 construct sequence for S30 experiments

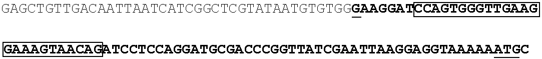

AAGTCGACCTGCTGGATCCAAAC (SEQ ID NO: 30)
Note:
40 bases from the 5’ terminal end indicate Tac promoter sequence. Bold letters indicate the sequences constituting the riboswitch. The underlined G indicates the transcription starting position, and the underlined ATG indicates the translation starting position. The box indicates the base sequence of SEQ ID NO: 1.

TABLE 12
pT7-H2 riboswitch -αHL

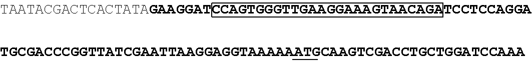

CATGGATTCTGATATCAATATCAAAACCGGCACCACCGATATCGGCTCCAATACCACCGTTAA
AACCGGTGATCTGGTGACCTATGATAAAGAAAACGGTATGCATAAAAAAGTGTTTTACTCGTT
TATTGACGATAAAAACCATAACAAAAAACTGCTGGTCATCCGCACCAAAGGCACCATTGCGG
GTCAATACCGTGTGTACTCCGAAGAAGGTGCGAACAAAAGCGGTCTGGCTTGGCCGTCTGCC
TTTAAAGTGCAGCTGCAACTGCCGGATAATGAAGTGGCGCAGATTTCAGATTATTATCCGCGT
AATAGCATCGATACCAAAGAATATATGAGTACCCTGACCTATGGTTTTAATGGCAATGTTACC
GGTGATGATACGGGTAAAATTGGCGGTCTGATTGGCGCCAATGTGTCCATTGGTCATACGCT
GAAATACGTGCAACCGGATTTCAAAACCATTCTGGAAAGTCCGACCGATAAAAAAGTGGGTT
GGAAAGTtATCTTCAACAACATGGTGAATCAGAACTGGGGTCCGTACGATCGCGATTCCTGGA
ATCCGGTTTATGGCAATCAGCTGTTTATGAAAACCCGCAACGGTAGTATGAAAGCGGCGGAT
AATTTTCTGGACCCGAAGAAAGGCTCAAGCCTGCTGTCCAGCGGTTTTAGCCCGGATTTTGC
CACGGTTATTACCATGGATCGCAAAGCCAGCAAACAGCAGACCAACATTGATGTGATCTACGA
ACGTGTGCGTGATGATTATCAACTGCATTGGACCTCAACCAATTGGAAAGGCACCAATACCAA
AGATAAATGGACGGATCGCAGTTCAGAACGCTACAAAATTGATTGGGAAAAAGAAGAAATGA
CTAGCATAACCCCTCTCTAAACGGAGGGGTTTGAATTCTAT (SEQ ID NO: 31)
Note:
17 bases from the 5’ terminal end indicate T7 promoter sequence. Bold letters indicate the sequences constituting the riboswitch. Gray letters indicate the coding sequence of αHL(α-hemolysin).The underlined G indicates the transcription starting position, and the underlined ATG indicates the translation starting position. The box indicates the base sequence of SEQ ID NO: 1.

### 2. Results

### 2.1. In Vitro Selection of Histamine-Binding RNA Aptamers

Histamine aptamer was selected from a pool of 2×10¹⁵ RNA molecules containing up to 3×10¹⁴ unique sequences within the 40-nt randomized region flanked by two 23-nt constant sequences. As histamine is a small compound with few functionalities, we immobilized L-histidine via its carboxyl group so that both the imidazole ring and the primary amine group would be available for the potential aptamers to interact. Stringency of the selection was gradually increased by increasing the number of washing steps, introduction of negative selection against L-histidine (from round 8), and lowering the histamine concentration in the elution buffer (from round 9) (Table 2). RNA populations before rounds 8, 9, and 11 were analyzed by deep sequencing together with elutions from these selection rounds by histamine as well as L-histidine to identify histamine-specific aptamer candidates.

Careful analysis of the sequencing results led us to A1 as a particularly promising candidate. A1 was found to be the second most abundant sequence in the histamine eluent from round 11, constituting 17.2% of the sequenced population. On the other hand, A1 comprised 5.8% in the L-histidine elution pool, indicating the preference of A1 to bind histamine. None of the 12 other sequences with abundance >0.5% in the histamine eluent from round 11 showed comparable preference for histamine over L-histidine. Furthermore, abundance of A1 progressively increased from 0.012% at the start of round 8, to 0.18%, and 10.98% at rounds 9 and 11, respectively. It has been observed that enrichment of a sequence through multiple rounds of SELEX is a more predictive indicator of aptamer affinity than absolute abundance in the last selection round (Schutze et al. 2011). Consequently, we selected A1 for further analysis.

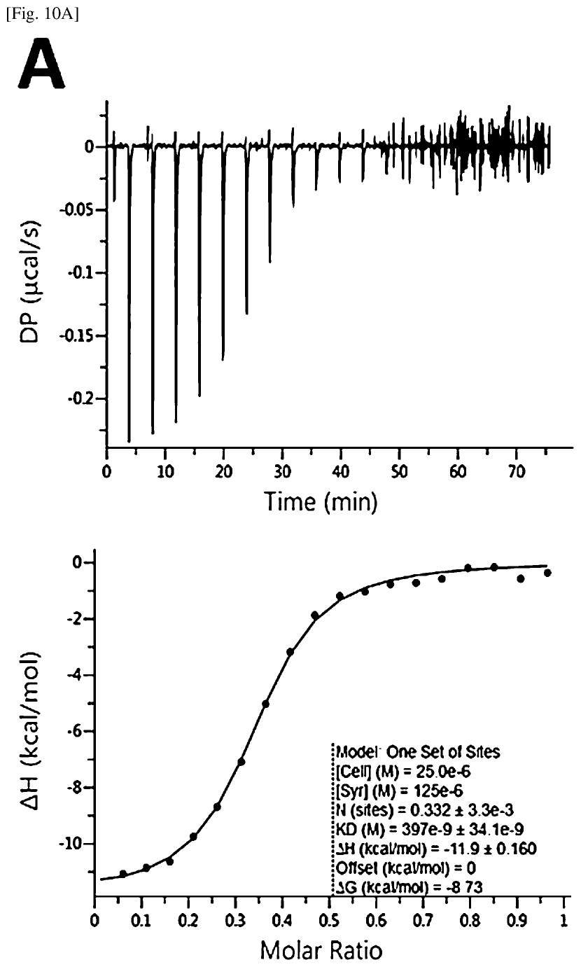

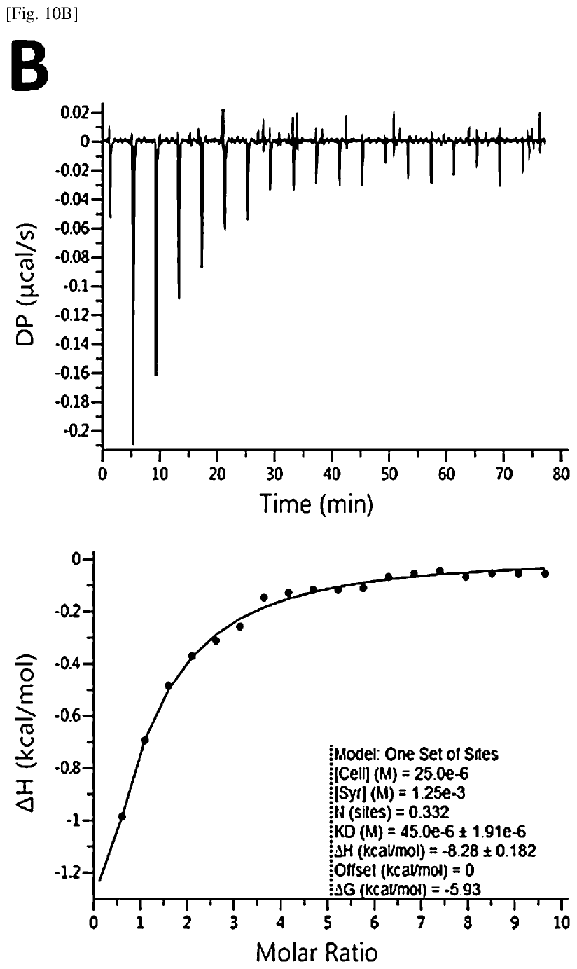

Full A1 sequence was synthesized by in vitro transcription and characterized by isothermal titration calorimetry (ITC). A1 showed a dissociation constant of (K_{d}) of 0.4 μM for histamine and 45 μM for L-histidine respectively (FIG. 10), confirming its high affinity for histamine and selectivity against L-histidine.

### 2.2. Further Characterization and Minimization of A1 Aptamer

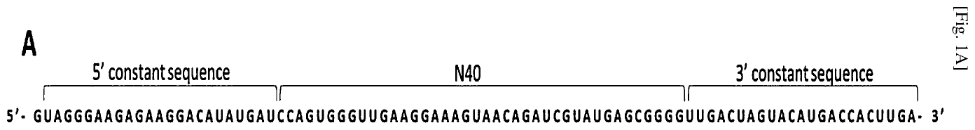

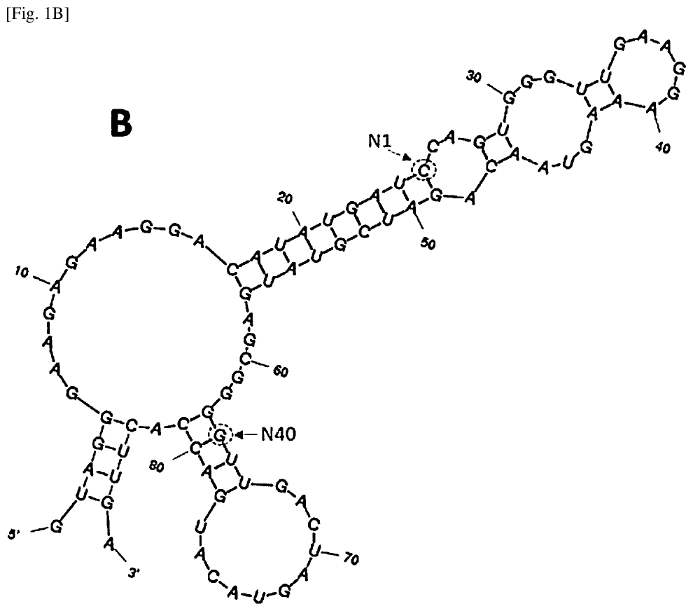

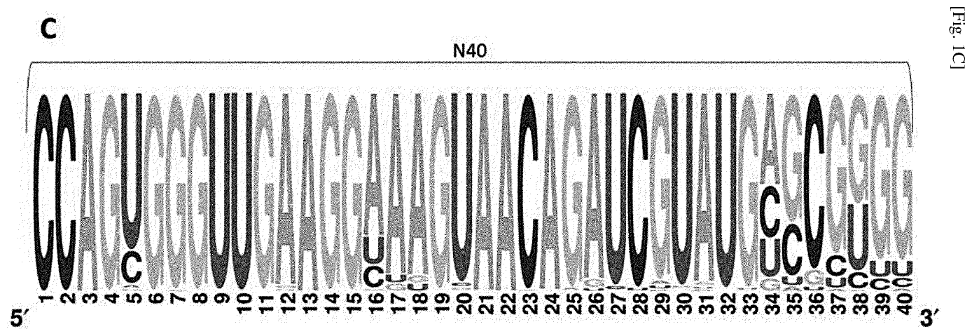

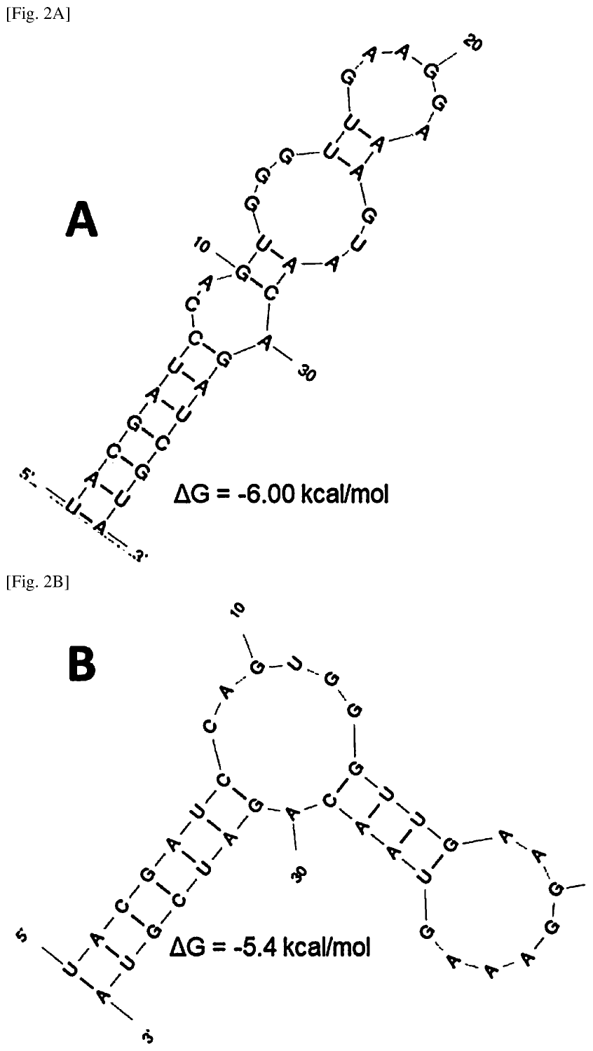

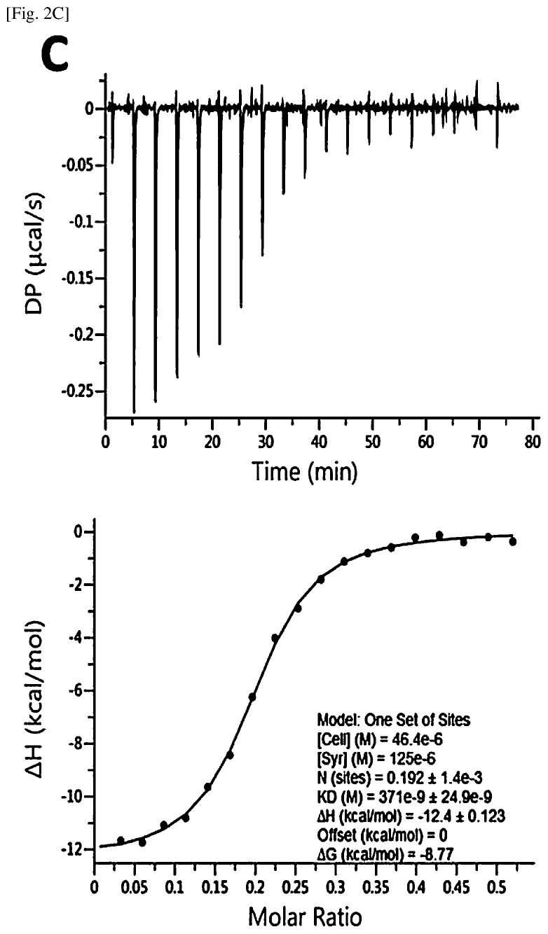

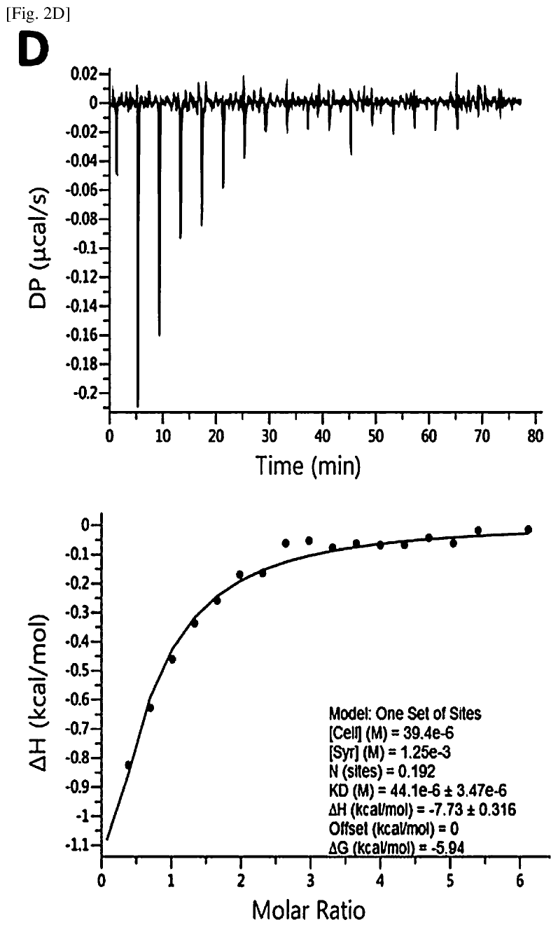

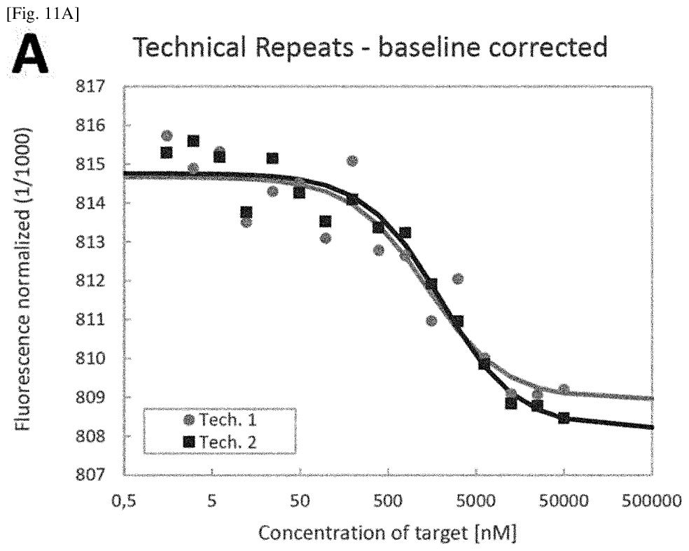

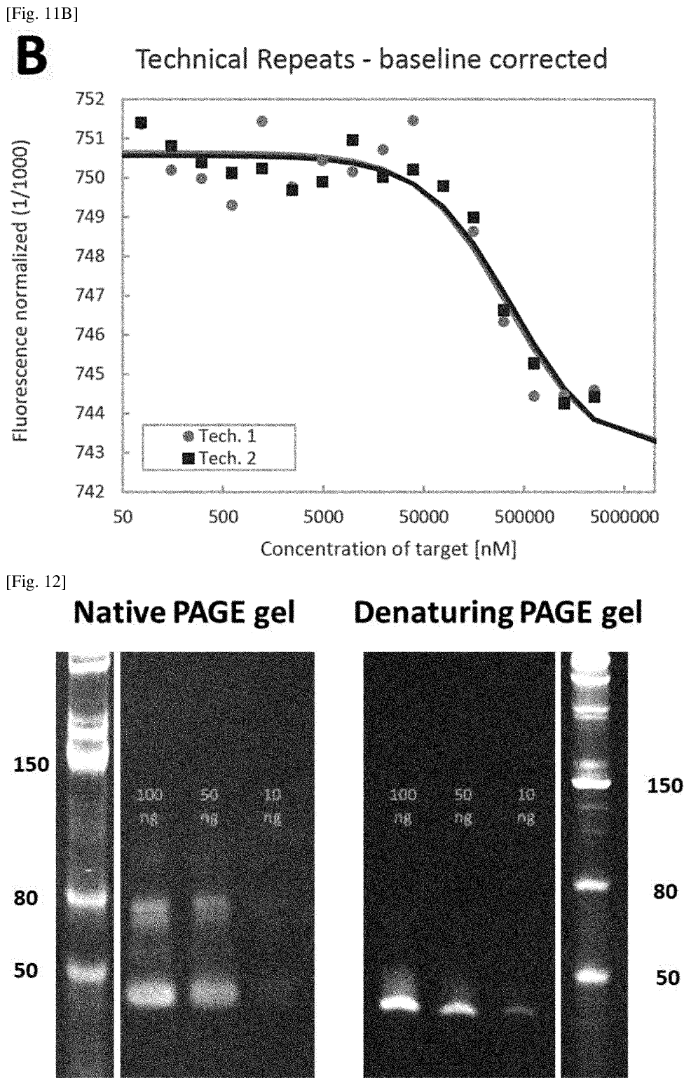

To identify the core sequence of the aptamer necessary for histamine binding, a partially randomized library of A1 aptamer was prepared by doping the selected bases at a rate of 21%. The library was subjected to four rounds of stringent selection for histamine binding and sequenced. The sequences which were enriched more than 50-fold after four rounds were analyzed to determine the variability of each nucleotide in the aptamer sequence (FIG. 1C). Bases from C1 through G33 were generally well conserved with some variability observed at U5, A16, A17, A18, and A26. On the other hand, A34 through G40 were highly variable, indicating that these bases are not involved in histamine binding. These observation and the predicted secondary structure of the A1 sequence by mfold (Zuker 2003) (FIG. 1B) led us to A1-949 as a minimal aptamer (FIG. 2A, and B). ITC measurement of A1-949 yielded similar dissociation constants (K_{d}) for histamine (0.37 μM) and L-histidine (44 μM) to those of A1 (FIG. 2C, and D). The stoichiometry (N) was significantly lower than 1.0 which can be due to several reasons. Native gel electrophoresis of the A1 aptamer showed multiple bands (FIG. 12) suggesting that the aptamer may exist in multiple secondary structures or multimeric complexes, thereby effectively lowering the active aptamer concentration in the ITC chamber. The high concentration (25-50 μM) needed for ITC measurements further increases the likelihood of formation of intermolecular complexes. Similar deviations from the ideal stoichiometry in small molecule binding RNA aptamers have been reported previously (Wu et al. 2015). Cy5-labeled A1-949 was used to measure the aptamer affinity by microscale thermophoresis (MST) which yielded similar K_{d} values of 1.7 μM and 362 μAM for histamine and L-histidine, respectively (FIG. 11). Evaluation of A1-949′s affinity for histamine and its selectivity against L-histidine by two distinct methods further validates the remarkable performance of the aptamer.

### 2.3. Developing Specific Histamine-Responsive Aptasensor

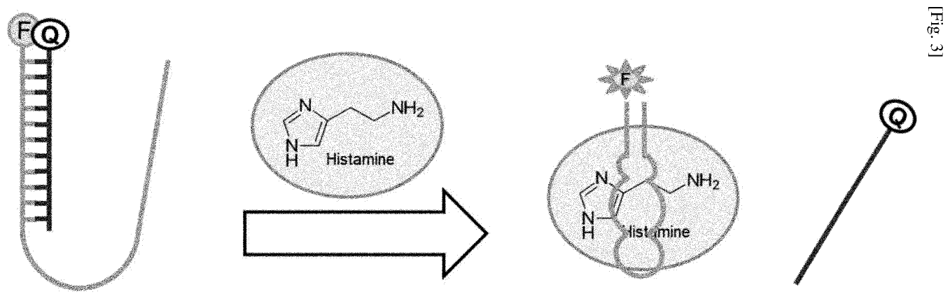

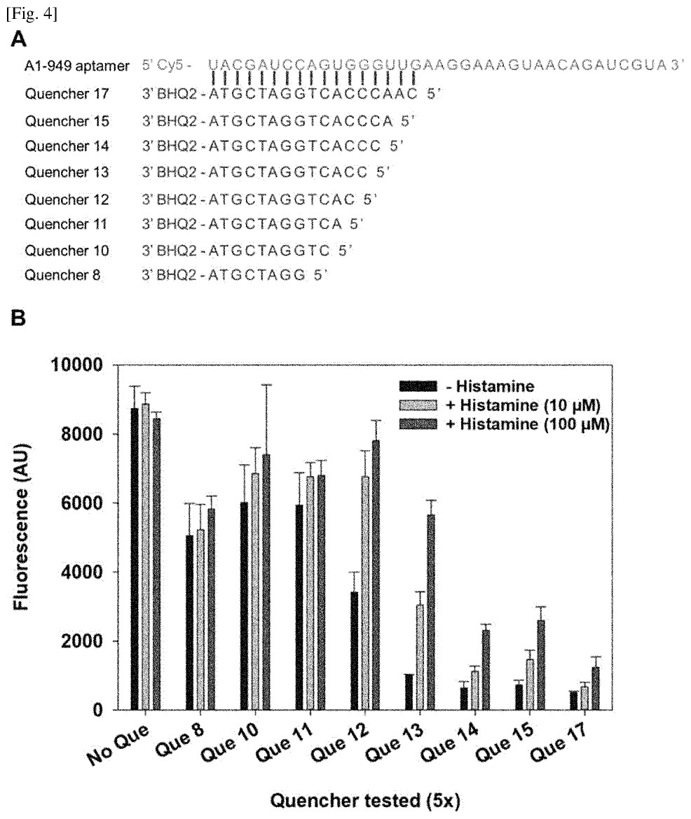

With the A1-949 aptamer in hand, we next decided to design a selective aptasensor for histamine. The general design strategy is shown in FIG. 3 which is based on the structure switching mechanism (Huang and Liu 2010; Nutiu and Li 2004). Briefly, the A1-949 aptamer was chemically synthesized with Cy5 fluorophore attached at the 5′ terminus (Cy5-A1-949) (FIG. 4A). DNA sequences that are complementary to the 5′ side of A1-949 with varying lengths were synthesized with a quencher (BHQ2) attached at the 3′ terminus (QueN: N=8, 10-15, 17) (FIG. 4A). Modified oligonucleotides Cy5-A1-949 and QueN were mixed with or without histamine and denatured by heating (65° C. for 20 min), and the solutions were allowed to cool to room temperature. Binding of histamine to Cy5-A1-949 was expected to inhibit its hybridization with the quencher-modified DNA (QueN), therefore resulting in increased fluorescence (FIG. 3).

As shown in FIG. 4, under the experimental conditions used (Quencher/aptamer molar ratio of 5, MgCl₂ 0.1 mM, and NaCl 250 mM), quencher 13 showed the best fluorescence response with ON/OFF ratio of 3- and 5.5-fold for 10 μM, and 100 μM histamine respectively, with reasonably low background expression.

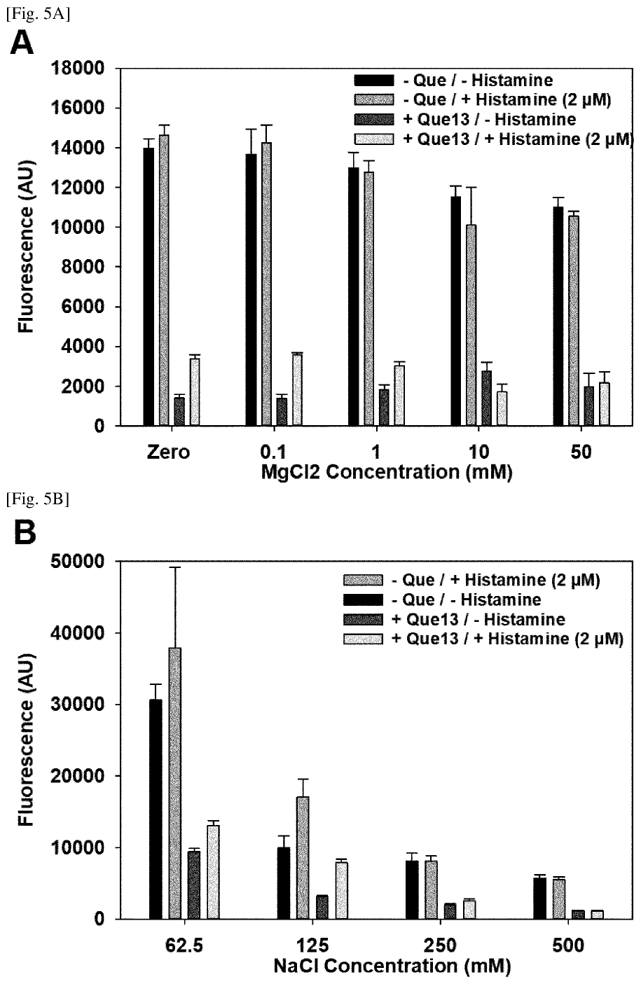

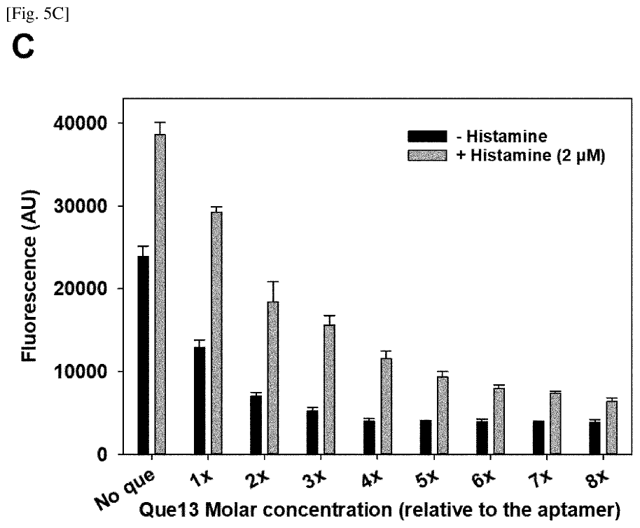

Further experiments were performed to optimize the assay buffer composition (FIG. 5). Interestingly, the aptasensor performance was found to be independent of Mg²⁺ concentration below 1 mM. This lack of Mg²⁺ requirement may be favorable for future applications of this aptamer in living cells where the concentration of free Mg²⁺ is known to be in the range of 1 mM or less (Romani 2011). Further optimizations were conducted for NaCl concentration and quencher : aptamer ratio (FIG. 5B, and C) which were determined to be 125 mM and 4:1, respectively.

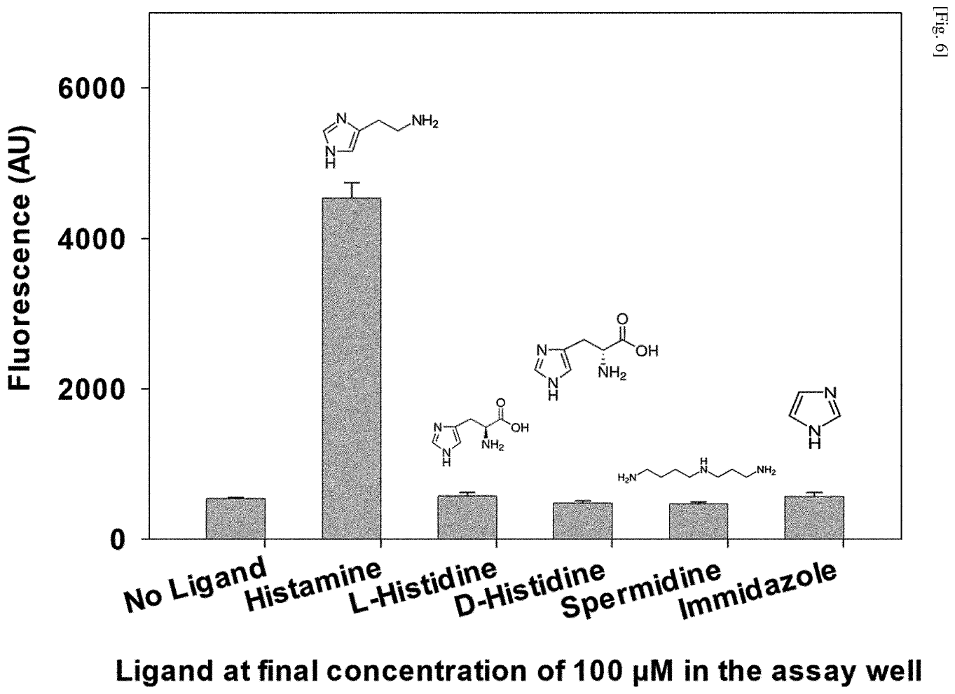

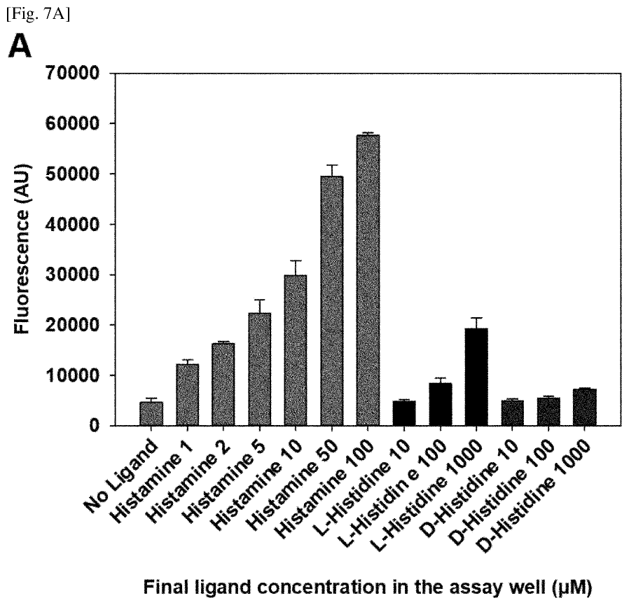

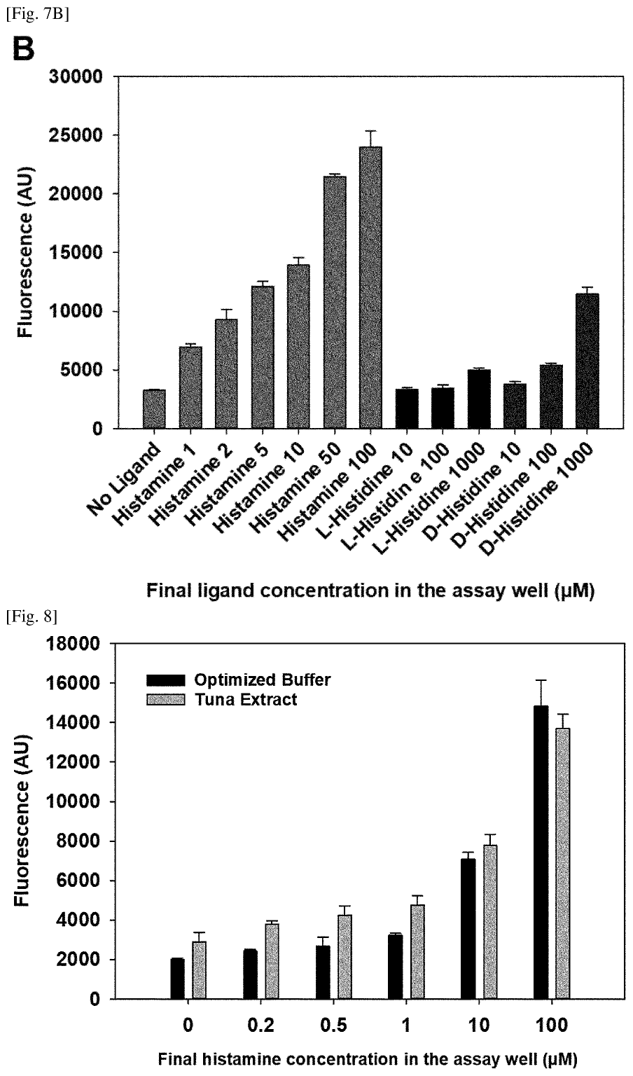

Specificity of the aptasensor under the optimized assay conditions was evaluated by observing its responses to a range of biochemically relevant compounds. None of the compounds tested: L-histidine, D-histidine, spermidine, and imidazole, showed appreciable response at 100 μM (FIG. 6). The sensor performance was further evaluated using various concentrations of histamine and histidine stereoisomers (FIG. 7A). The aptasensor could detect histamine concentration as low as 1 μM. Although the aptasensor displayed an appreciable response at 1000 μM L-histidine, the signal was lower than that of histamine at 5 μM. Moreover, the aptasensor showed negligible response to D-histidine even at 1000 μM (FIG. 7A).

### 2.4. Minor-Image Aptasensor Displays Improved Selectivity Against L-Histidine

RNA is highly susceptible to ribonucleases which are widely found in environmental and biological samples. Therefore, aptasensors based on RNA aptamers are limited in their practical applications. Since histamine is achiral, however, an aptamer entirely composed of L-ribonucleotides should display an identical affinity to histamine. Such L-RNA aptamers are known to be highly resistant to ribonucleases (Eulberg and Klussmann 2003). Therefore, the A1-949 aptamer sequence was synthesized using L-ribonucleotides modified with a Cy3 fluorophore at the 5′ end (Cy3-A1-949*). Corresponding quencher DNA (Que13*) was also synthesized using L-deoxynucleotides with a BHQ2 modification at the 3′ terminus. The mirror-image (L-form) aptasensor was evaluated using histamine and histidine stereoisomers (FIG. 7B). As expected, the response to histamine by the L-form aptasensor was comparable to that of the original sensor. Furthermore, the mirror-image aptasensor showed negligible response to L-histidine as expected from the inversion of the sensor's stereochemistry. Although this was an unintended consequence, the improved selectivity against the more abundant L-histidine improves the practical utility of the aptasensor (FIG. 7B).

### 2.5. Quantification of Histamine in Spiked Fish Samples

The L-form aptasensor was evaluated by measuring fish samples spiked with known concentrations of histamine. We chose fresh tuna meat which is frequently associated with histaminosis and routinely screened for possible histamine contamination (Feng et al. 2016). Raw tuna pieces were spiked with various histamine concentrations, heated, homogenized, and deproteinated by ultrafiltration. The extracted histamine samples were assayed by the L-form aptasensor as described above. The results (FIG. 8) indicate that the aptasensor shows comparable responses to spiked samples in both tuna extract and in buffer albeit with slightly higher background in the tuna extract.

### 2.6. Developing Histamine-Responsive Riboswitch

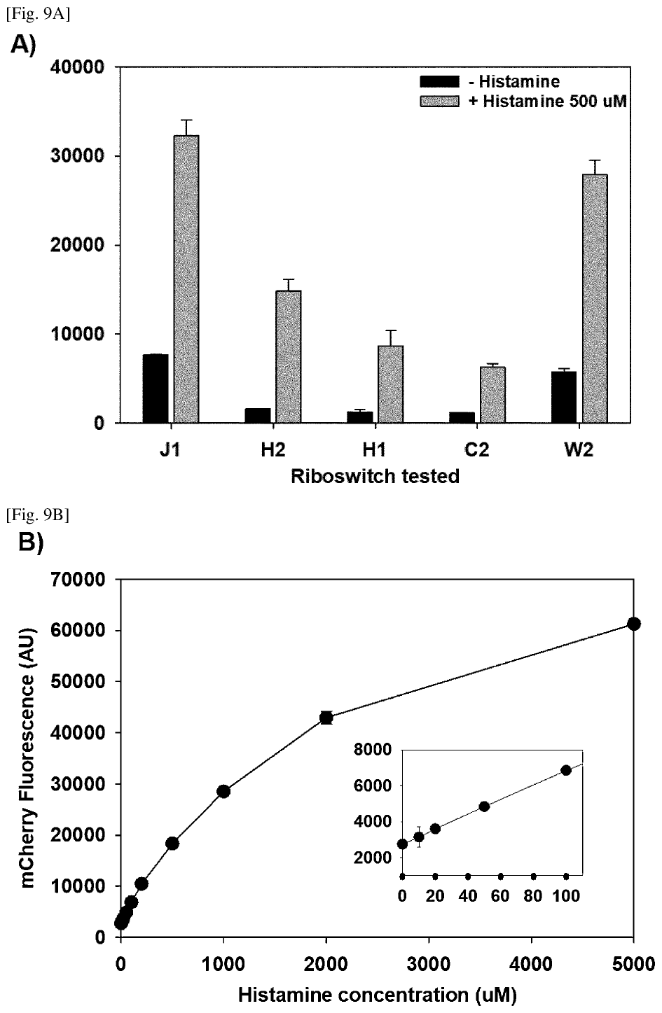

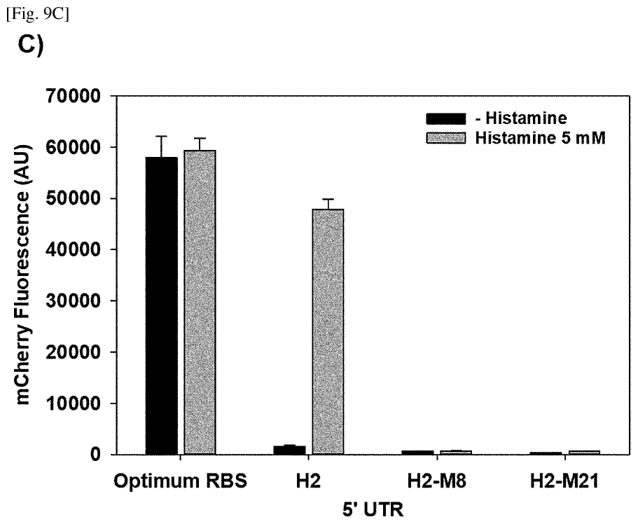

With the A1-949 aptamer in hand, we then opt to utilize it to build a synthetic riboswitch. Histamine is a polar molecule which is not likely to diffuse through the bacterial cell membrane. In fact, some bacterial strains export histamine, which is made intracellularly, through membrane-bound antiporter in exchange for histidine (Kimura et al. 2009). Consequently, we were not able to perform in -vivo screening that is typically used for developing RNA riboswitches from aptamers (Nomura and Yokobayashi 2014). In contrast, we decided to use a combination of rational design and test tube screening using the PURE transcription/translation system (Shimizu et al. 2005). After several rounds of screening and optimization, we got a series of successful riboswitches with various sensitivities to histamine (FIG. 9A). The best riboswitch (H2) was then tested further to determine its dynamic range (FIG. 9B). This riboswitch was able to give ON/OFF ratio of 30.7-fold (FIG. 9C), albeit at relatively high histamine concentration (5 mM).

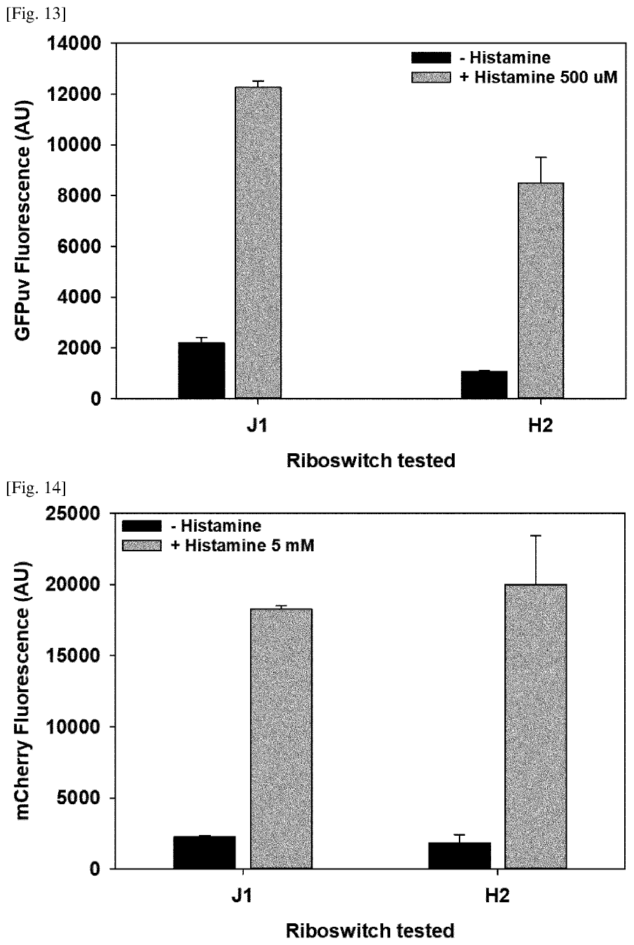

To confirm that the switching response seen is due to conformation change in the A1-949 aptamer upon binding to histamine and not just a general effect of histamine on the transcription or the translation machinery, experiments were also done using mutated H2 riboswitch as well as with mCherry genetic construct that has optimum RBS sequence (As obtained from the RBS calculator program (Salis et al. 2009, Borujeni et al. 2014). As expected, the mutations made in the aptamer region significantly decreased or even abolished the signaling activity (FIG. 9C). Furthermore, to demonstrate the applicability to other proteins, mCherry was replaced with GFP (FIG. 13) and the results found also similar switching activities. Similarly, to show the possible application of these riboswitches in another commercial in-vitro Transcription/translation systems, J1 and H2 riboswitches were tested in the linear S30 system (Promega) after replacing the T7 promoter for Tac promoter. The results (FIG. 14) found again comparable activities to those seen in the PURE system.

### 3. Discussion

Although numerous strategies have been developed to turn aptamers into sensors, availability of aptamers with suitable affinity and specificity continues to be the limiting factor in adapting the technologies to new molecular targets. Small molecules are especially challenging targets as aptamer ligands, because they display fewer functionalities with which the potential aptamers can interact compared to proteins. In this work, we selected an RNA aptamer with high affinity and specificity for histamine, a metabolite involved in a number of biological processes including food poisoning. Deep sequencing allowed us to identify an aptamer with exquisite selectivity against histidine, a probable contaminant present in many biological samples. We further demonstrated the practical utility of the histamine aptamer by designing an aptasensor with fluorescence readout by synthesizing the sensor oligonucleotides using nucleaseresistant L-form nucleotides. The RNA aptamer we described may also have other biological applications, for example, as riboswitches to control gene expression in living cells (REF).

This aptamer was then successfully converted into synthetic riboswitch which can specifically respond to histamine in bulk solution.

The sensitivity of the riboswitches made in the current study is above the physiological range of histamine. However, as the dissociation constant (Kd) of the aptamer is in the nanomolar range, it should be possible in the future to employ this aptamer to develop riboswitches with better sensitivity. Furthermore, the sensitivity of the riboswitch can be greatly improved through applying a positive feedback loop as previously described by Kobori et al (Kobori et al. 2013).

Aside from the potential use of the developed histamine riboswitches in artificial liposomes, they can also be applied to study the ability of different bacterial strains especially from the gut microbiome to import histamine. In fact, a recent report suggested that bacteria such as Lactobacillus salivarius express homologues for both the mammalian plasma membrane monoamine transporter (PMAT) and organic cation transporter (OCT) (Lyte and Brown 2018). The availability of a histamine-responsive riboswitch, in turn can help in studying these phenomena. Finally, the histamine aptamer developed here could be also applied in the future as a sensitive and selective biosensor for the detection and quantification of histamine in food products (Feng et al. 2016) as well as other biological samples for diagnostic purposes (Laroche et al. 1991, Platzer et al 2005).

### REFERENCES

Codrea, V., Hayner, M., Hall, B., Jhaveri, S., Ellington, A., 2010. In vitro selection of RNA aptamers to a small molecule target. Current protocols in nucleic acid chemistry Chapter 9, Unit 9 5 1-23.

Eulberg, D., Klussmann, S., 2003. Spiegelmers: Biostable aptamers. Chembiochem 4(10), 979-983.

Feng, C., Teuber, S., Gershwin, M. E., 2016. Histamine (Scombroid) Fish Poisoning: a Comprehensive Review. Clinical reviews in allergy & immunology 50(1), 64-69.

Hu, J., Chen, T., Mingkal, L., He, G., Meng, J., Ma, X., Wu, Y., Jia, M., Luo, X., 2007. Wide distribution and subcellular localization of histamine in sympathetic nervous systems of different species. Neurosci Res 59(2), 231-236.

Huang, P. J. J., Liu, J. W., 2010. Flow Cytometry-Assisted Detection of Adenosine in Serum with an Immobilized Aptamer Sensor. Anal Chem 82(10), 4020-4026.

Illangasekare, M., Yarus, M., 2002. Phenylalanine-binding RNAs and genetic code evolution. Journal of molecular evolution 54(3), 298-311.

Janci, T., Valinger, D., Kljusuric, J. G., Mikac, L., Vidacek, S., Ivanda, M., 2017. Determination of histamine in fish by Surface Enhanced Raman Spectroscopy using silver colloid SERS substrates. Food Chem 224, 48-54.

Kielland, N., Vendrell, M., Lavilla, R., Chang, Y. T., 2012. Imaging histamine in live basophils and macrophages with a fluorescent mesoionic acid fluoride. Chem Commun 48(59), 7401-7403.

Laroche, D., Vergnaud, M. C., Sillard, B., Soufarapis, H., Bricard, H., 1991. Biochemical markers of anaphylactoid reactions to drugs. Comparison of plasma histamine and tryptase. Anesthesiology 75(6), 945-949.

Majerfeld, I., Puthenvedu, D., Yarus, M., 2005. RNA affinity for molecular L-histidine; genetic code origins. Journal of molecular evolution 61(2), 226-235.

McKeague, M., Derosa, M. C., 2012. Challenges and opportunities for small molecule aptamer development. Journal of nucleic acids 2012, 748913.

Muscarella, M., Lo Magro, S., Campaniello, M., Armentano, A., Stacchini, P., 2013. Survey of histamine levels in fresh fish and fish products collected in Puglia (Italy) by ELISA and HPLC with fluorimetric detection. Food Control 31(1), 211-217.

Nei, D., Nakamura, N., Ishihara, K., Kimura, M., Satomi, M., 2017. A rapid screening of histamine concentration in fish fillet by direct analysis in real time mass spectrometry (DART-MS). Food Control 75, 181-186.

Nielsen, O. H., Rask-Madsen, J., 1996. Mediators of inflammation in chronic inflammatory bowel disease. Scandinavian journal of gastroenterology. Supplement 216, 149-159.

Nutiu, R., Li, Y. F., 2004. Structure-switching signaling aptamers: Transducing molecular recognition into fluorescence signaling. Chem-Eur J 10(8), 1868-1876.

Onal, A., 2007. A review: Current analytical methods for the determination of biogenic amines in foods. Food Chem 103(4), 1475-1486.

Pessatti, T. L., Fontana, J. D., Pessatti, M. L., 2004. Spectrophotometric determination of histamine in fisheries using an enzyme immunoassay method. Methods in molecular biology 268, 311-316.

Pfeiffer, F., Mayer, G., 2016. Selection and Biosensor Application of Aptamers for Small Molecules. Frontiers in chemistry 4, 25.

Platzer, M. H., Grattan, C. E., Poulsen, L. K., Skov, P. S., 2005. Validation of basophil histamine release against the autologous serum skin test and outcome of seruminduced basophil histamine release studies in a large population of chronic urticaria patients. Allergy 60(9), 1152-1156.

Romani, A. M., 2011. Cellular magnesium homeostasis. Archives of biochemistry and biophysics 512(1), 1-23.

Ruscito, A., DeRosa, M. C., 2016. Small-Molecule Binding Aptamers: Selection Strategies, Characterization, and Applications. Frontiers in chemistry 4, 14.

Sato, T., Horiuchi, T., Nishimura, I., 2005. Simple and rapid determination of histamine in food using a new histamine dehydrogenase from Rhizobium sp. Anal Biochem 346(2), 320-326.

Schutze, T., Wilhelm, B., Greiner, N., Braun, H., Peter, F., Morl, M., Erdmann, V. A., Lehrach, H., Konthur, Z., Menger, M., Arndt, P. F., Glokler, J., 2011. Probing the SELEX Process with Next-Generation Sequencing. Plos One 6(12).

Seto, D., Soh, N., Nakano, K., Imato, T., 2010. An amphiphilic fluorescent probe for the visualization of histamine in living cells. Bioorg Med Chem Lett 20(22), 6708-6711.

Wilson, D. S., Keefe, A. D., 2001. Random mutagenesis by PCR. Current protocols in molecular biology Chapter 8, Unit8 3.

Wu, M. C., Lowe, P. T., Robinson, C. J., Vincent, H. A., Dixon, N., Leigh, J., Micklefield, J., 2015. Rational Re-engineering of a Transcriptional Silencing PreQ1 Riboswitch. Journal of the American Chemical Society 137(28), 9015-9021.

Zuker, M., 2003. Mfold web server for nucleic acid folding and hybridization prediction. Nucleic Acids Res 31(13), 3406-3415.

Dwidar, M., and Yokobayashi, Y., 2017. Controlling Bdellovibrio bacteriovorus Gene Expression and Predation Using Synthetic Riboswitches, Acs Synth Biol 6, 2035-2041. Kimura, B., Takahashi, H., Hokimoto, S., Tanaka, Y., and Fujii, T., 2009. Induction of the histidine decarboxylase genes of Photobacterium damselae subsp damselae (formally P. histaminum) at low pH, J Appl Microbiol 107, 485-497.

Nomura, Y., and Yokobayashi, Y., 2014. Dual genetic selection of synthetic riboswitches in Escherichia coli, Methods in molecular biology 1111, 131-140.

Shimizu, Y., Kanamori, T., and Ueda, T. 2005. Protein synthesis by pure translation systems, Methods 36, 299-304.

Salis, H. M., Mirsky, E. A., and Voigt, C. A., 2009. Automated design of synthetic ribosome binding sites to control protein expression, Nat Biotechnol 27, 946-U112.

Borujeni, A. E., Channarasappa, A. S., and Salis, H. M., 2014. Translation rate is controlled by coupled trade-offs between site accessibility, selective RNA unfolding and sliding at upstream standby sites, Nucleic Acids Res 42, 2646-2659.

Kobori, S., Ichihashi, N., Kazuta, Y., and Yomo, T., 2013. A controllable gene expression system in liposomes that includes a positive feedback loop, Mol Biosyst 9, 1282-1285.

Lyte, M., and Brown, D. R., 2018. Evidence for PMAT- and OCT-like biogenic amine transporters in a probiotic strain of Lactobacillus: Implications for interkingdom communication within the microbiota-gut-brain axis, Plos One 13.

1. A nucleic acid aptamer that binds to histamine, comprising the base sequence (i) or (ii) below:
(i) the base sequence of SEQ ID NO: 1
(5′-CCAGUGGGUUGAAGGAAAGUAACAG-3′)
(ii) the base sequence comprising substitution(s), deletion(s), and/or addition(s) of 1 to 3 base(s) in the base sequence of SEQ ID NO: 1.

2. The nucleic acid aptamer according to claim 1, wherein base sequences that can form a double-stranded stem structure through complementary base pairing are added to both ends of the base sequence (i) or (ii).

3. The nucleic acid aptamer according to claim 2, wherein stem base sequence 1 (5′-UACGAU-3′) is added to the 5′ end or the 3′ end of the base sequence (i) or (ii) and stem base sequence 2 (5′-AUCGUA-3′) is added to the other end.

4. The nucleic acid aptamer according to claim 1, wherein substitution(s) is/are made at least one positon of position 5 (U), position 16 (A), position 17 (A) and position 18 (A) of SEQ ID NO: 1.

5. The nucleic acid aptamer according to claim 1, wherein the binding force to histamine is higher than the binding force to histidine.

6. The nucleic acid aptamer according to claim 1, wherein the dissociation constant for histamine is no more than 5 μM.

7. The nucleic acid aptamer according to claim 1, wherein the dissociation constant for histidine is no less than 40 μM.

8. The nucleic acid aptamer according claim 1, wherein the nucleic acid is at least one selected from the group consisting of DNA, RNA, and artificial nucleic acid.

9. The nucleic acid aptamer according to claim 8, wherein the nucleic acid is at least one selected from the group consisting of L-DNA and L-RNA.

10. A composition for histamine detection comprising the nucleic acid aptamer according to claim 1.

11. A histamine detection kit comprising the nucleic acid aptamer according to claim 1.

12. A biosensor for detecting histamine, comprising the nucleic acid aptamer according to claim 1.

13. A method for detecting histamine in a subject sample, which method is characterized by using the nucleic acid aptamer according to claim 1.

14. A method for detecting histamine in a subject sample, which comprises
preparing a complex having a double strand formed of the nucleic acid aptamer according to claim 1 and a nucleic acid fragment having a base sequence complementary to a part of the nucleic acid aptamer;
separating the nucleic acid fragment from the nucleic acid aptamer by binding histamine in subject sample with the nucleic acid aptamert; and
detecting cleavage of the double strand by the above separation.

15. The method for detecting histamine according to claim 14, wherein a fluorescent substance is bound to the nucleic acid aptamer and a quencher substance is bound to the nucleic acid fragment.

16. A riboswitch comprising the nucleic acid aptamer according to claim 1.

17. The riboswitch according to claim 16, which comprises any of the base sequences of SEQ ID NOs: 2 to 6.
(SEQ ID NO: 2)
5′-GAAGGAUCCAGUGGGUUGAAGGAAAGUAACAGAUCCUCCAGGAUGC
GACCCGGUUAUCGAAUUAAGGAGGUAAAAAAUGCAAGUCGACCUGCUGG
AUCCAAAC-3′
(SEQ ID NO: 3)
5′-GAAGGAUCCAGUGGGUUGAAGGAAAGUAACAGAUCCUUUAGGAUGC
GACCCGGUUAUCGAAUUAAGGAGGUAAAAAAUGCAAGUCGACCUGCUGG
AUCCAAAC-3′
(SEQ ID NO: 4)
5′-GAAGGAUCCAGUGGGUUGAAGGAAAGUAACAGAUCCUCCAGGAUGC
GACCCGGUUAUCGAAUUAAGGAGGUAAAAAAUGCAAGUCGACCUGCUCG
AUCCAAAC-3′
(SEQ ID NO: 5)
5′-GAGGAUCCAGUGGGUUGAAGGAAAGUAACAGAUCCUUCAGGAUGCG
ACCCGGUUAUCGAAUUAAGGAGGUAAAAAAUGCAAGUCGACCUGCUGGA
UCCAAAC-3′
(SEQ ID NO: 6)
5′-GACGAUCCAGUGGGUUGAAGGAAAGUAACAGAUCGUUCAGGAUGCG
ACGCGCUUGGAAUUAAGGAGGUAAAAAAUGCAAGUCGACCUGCUGGAUC
CAAAC-3′

18. A RNA polynucleotide comprising a regulation region corresponding to the riboswitch according to claim 16 and a cording region for a protein, wherein the expression of the protein is regulated by the regulation region.

19. A nucleic acid construct comprising a promoter sequence and a base sequence complementary to the RNA polynucleotide according to claim 18 operably linked thereto.
